SEQUENCE LISTING (31 sequences)

SEQ ID NO: 1 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 132 aa, synthetic construct
PASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 132 aa, synthetic construct
PTSSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - protein, 132 aa, synthetic construct
PASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 226 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 226 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 226 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 8 - protein, 10 aa, synthetic construct
GGGGSGGGGS

SEQ ID NO: 9 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 368 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSPASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 12 - protein, 368 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSPASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 13 - protein, 133 aa, Homo sapiens [sequence omitted]

SEQ ID NO: 14 - protein, 132 aa, Homo sapiens [sequence omitted]

SEQ ID NO: 15 - protein, 8 aa, synthetic construct
GGGSGGGS

SEQ ID NO: 16 - protein, 12 aa, synthetic construct
GGGGGSGGGGGS

SEQ ID NO: 17 - protein, 369 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSPASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 18 - protein, 132 aa, synthetic construct
PTSSSTKKTQLQLEHLLLDLQMILNGINNYKNPKLTQMLTFKFFMPKKATELKHLQCLEEELKPLEEVLNLAQSKNFHLRPRDLISNINVIVLELKGSETTFMCEYADETATIVEFLNRWITFSQSIISTLT

SEQ ID NO: 19 - protein, 132 aa, synthetic construct
PASSSTKKTQLQLEHLLLDLQMILNGINNYKNPKLTQMLTFKFFMPKKATELKHLQCLEEELKPLEEVLNLAQSKNFHLRPRDLISNINVIVLELKGSETTFMCEYADETATIVEFLNRWITFSQSIISTLT

SEQ ID NO: 20 - protein, 369 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGGSGGGGSPASSSTKKTQLQLEHLLLDLQMILNGINNYKNPKLTQMLTFKFFMPKKATELKHLQCLEEELKPLEEVLNLAQSKNFHLRPRDLISNINVIVLELKGSETTFMCEYADETATIVEFLNRWITFSQSIISTLT

SEQ ID NO: 21 - protein, 369 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSGGGGSPASSSTKKTQLQLEHLLLDLQMILNGINNYKNPKLTQMLTFKFFMPKKATELKHLQCLEEELKPLEEVLNLAQSKNFHLRPRDLISNINVIVLELKGSETTFMCEYADETATIVEFLNRWITFSQSIISTLT

SEQ ID NO: 22 - protein, 369 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSPASSSTKKTQLQLEHLLLDLQMILNGINNYKNPKLTQMLTFKFFMPKKATELKHLQCLEEELKPLEEVLNLAQSKNFHLRPRDLISNINVIVLELKGSETTFMCEYADETATIVEFLNRWITFSQSIISTLT

SEQ ID NO: 23 - protein, 5 aa, synthetic construct
GGGGS

SEQ ID NO: 24 - protein, 12 aa, synthetic construct
GSGGGGSGGGGS

SEQ ID NO: 25 - protein, 363 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSPASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 26 - protein, 366 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSPASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 27 - protein, 370 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGGGSGGGGSPASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 28 - protein, 364 aa, synthetic construct
APASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLTGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 369 aa, synthetic construct
APASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLTGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 363 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSPASSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 31 - protein, 368 aa, synthetic construct; identical to SEQ ID NO: 12

(19) United States
(12) Patent Application Publication
(10) Pub. No.: US 2024/0309061 A1
(43) Pub. Date: Sep. 19, 2024

(54) IL-2 MUTEINS FOR TREATING CANCER OR INFECTION

(71) Applicant: Merck Sharp & Dohme LLC, Rahway, NJ (US)

(72) Inventors: Juan C. Alvarez, Lincoln, MA (US); Alan C. Cheng, San Francisco, CA (US); Ghassan N. Fayad, Waban, MA (US); Brian E. Hall, Wellesley Hills, MA (US); Veronica M. Juan, Redwood City, CA (US); Peter S. Kim, Chestnut Hill, MA (US); Sheila H. Ranganath, Arlington, MA (US); Venkataraman Sriraman, Arlington, MA (US); Gary C. Starling, San Ramon, CA (US); Nicole C. Walsh, Medford, MA (US)

(73) Assignee: Merck Sharp & Dohme LLC, Rahway, NJ (US)

(21) Appl. No.: 18/578,343

(22) PCT Filed: Jul. 21, 2022
(86) PCT No.: PCT/US2022/037816

§ 371 Date: Jan. 11, 2024

Related U.S. Application Data
(60) Provisional application No. 63/225,065, filed on Jul. 23, 2021.

(51) Int. Cl. C07K 14/55; A61K 38/00; A61K 39/00; A61P 35/00; A61P 37/06; C07K 16/28

(52) U.S. Cl. CPC C07K 14/55; A61P 35/00; A61P 37/06; C07K 16/2818; C07K 16/2827; A61K 38/00; A61K 2039/505; C07K 2319/30

(57) ABSTRACT

Provided herein are IL-2 muteins that bind to the IL-2 receptor β subunit but do not have measurable binding to the IL-2 receptor a subunit. Also provided are compositions, kits, methods, and uses involving such IL-2 muteins.

### CROSS-REFERENCE TO RELATED APPLICATIONS

This application claims the benefit of U.S. Provisional Application No. 63/225,065, filed Jul. 23, 2021, the content of which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Jul. 20, 2022, is named 25267-WO-PCT-SL.XML and is 34KB bytes in size.

### FIELD

The invention relates to IL-2 muteins that bind to the IL-2 receptor β subunit but do not have measurable binding to the IL-2 receptor α subunit. The invention also relates to compositions, kits, methods, and uses involving such IL-2 muteins.

### BACKGROUND

The biological activity of interleukin-2 (IL-2) is mediated through a multi-subunit IL-2 receptor (IL-2R) complex comprising two or three polypeptide subunits: CD25 (IL-2R α subunit) enhances affinity of the IL-2R complex to IL-2, while CD122 (IL-2R β subunit) and CD132 (IL-2R γ subunit) are required for signal transduction. The dimeric IL-2R βγ complex and trimeric IL-2R αβγ complex are differentially expressed by various immune cell subtypes. For example, the high affinity trimeric IL-2R αβγ complex is constitutively expressed at high levels by regulatory T (Treg) cells and transiently expressed at lower levels by CD4+ T effector cells, whereas the moderate affinity dimeric IL-2R βγ complex is expressed primarily on CD8+ T cells and natural killer cells (Stauber et al., (2006) PNAS USA 103(8):2788-93; Malek and Castro, (2010) Immunity 33(2): 153-165; Ross and Cantrell, (2018) Annu Rev Immunol 36:418-433). Thus, IL-2 muteins with a bias to bind to the dimeric or trimeric IL-2R complex can activate different cell types and mediate either immune activation or suppression, respectively. There remains an unmet need for innovative IL-2 muteins that can more selectively activate IL-2R signals in distinct cell types to treat immune-mediated diseases, including biologically optimized IL-2 muteins selectively binding to the IL-2R β subunit for the treatment of cancer and infection.

### SUMMARY OF THE INVENTION

The present disclosure provides IL-2 muteins that bind to IL-2 receptor β subunit but do not have measurable binding to IL-2 receptor a subunit. Also provided herein are methods or uses involving such IL-2 muteins, compositions or kits comprising such IL-2 muteins, isolated nucleic acids and vectors comprising polynucleotide sequences encoding such IL-2 muteins, cells (e.g., host cells) comprising such isolated nucleic acids or vectors, and methods of producing such IL-2 muteins.

In one aspect, provided herein is an IL-2 mutein that binds to the IL-2 receptor β subunit but does not have measurable binding to the IL-2 receptor a subunit.

In certain embodiments, provided is an IL-2 mutein that comprises a first polypeptide comprising the amino acid sequence as set forth in SEQ ID NO: 3, 4, 18, or 19. In one embodiment, the IL-2 mutein comprises a first polypeptide comprising the amino acid sequence as set forth in SEQ ID NO:3. In another embodiment, the IL-2 mutein comprises a first polypeptide comprising the amino acid sequence as set forth in SEQ ID NO:4. In one embodiment, the IL-2 mutein comprises a first polypeptide comprising the amino acid sequence as set forth in SEQ ID NO:18. In another embodiment, the IL-2 mutein comprises a first polypeptide comprising the amino acid sequence as set forth in SEQ ID NO:19.

In some embodiments of the IL-2 muteins of the invention, the first polypeptide further comprises the amino acid sequence as set forth in SEQ ID NO: 5, 6, or 7. In one embodiment, the IL-2 mutein comprises a first polypeptide comprising the amino acid sequence as set forth in SEQ ID NO:3, 4, 18, or 19 and further comprising the amino acid sequence as set forth in SEQ ID NO:5, 6, or 7. In another embodiment, the IL-2 mutein comprises a first polypeptide comprising the amino acid sequence as set forth in SEQ ID NO:3 and further comprising the amino acid sequence as set forth in SEQ ID NO:5. In yet another embodiment, the IL-2 mutein comprises a first polypeptide comprising the amino acid sequence as set forth in SEQ ID NO:3 and further comprising the amino acid sequence as set forth in SEQ ID NO:6. In still another embodiment, the IL-2 mutein comprises a first polypeptide comprising the amino acid sequence as set forth in SEQ ID NO:3 and further comprising the amino acid sequence as set forth in SEQ ID NO:7. In yet still another embodiment, the IL-2 mutein comprises a first polypeptide comprising the amino acid sequence as set forth in SEQ ID NO:4 and further comprising the amino acid sequence as set forth in SEQ ID NO:5. In another embodiment, the IL-2 mutein comprises a first polypeptide comprising the amino acid sequence as set forth in SEQ ID NO:4 and further comprising the amino acid sequence as set forth in SEQ ID NO:6. In yet another embodiment, the IL-2 mutein comprises a first polypeptide comprising the amino acid sequence as set forth in SEQ ID NO:4 and further comprising the amino acid sequence as set forth in SEQ ID NO:7. In another embodiment, the IL-2 mutein comprises a first polypeptide comprising the amino acid sequence as set forth in SEQ ID NO:18 and further comprising the amino acid sequence as set forth in SEQ ID NO:5. In yet another embodiment, the IL-2 mutein comprises a first polypeptide comprising the amino acid sequence as set forth in SEQ ID NO:18 and further comprising the amino acid sequence as set forth in SEQ ID NO:6. In still another embodiment, the IL-2 mutein comprises a first polypeptide comprising the amino acid sequence as set forth in SEQ ID NO:18 and further comprising the amino acid sequence as set forth in SEQ ID NO:7. In yet still another embodiment, the IL-2 mutein comprises a first polypeptide comprising the amino acid sequence as set forth in SEQ ID NO:19 and further comprising the amino acid sequence as set forth in SEQ ID NO:5. In another embodiment, the IL-2 mutein comprises a first polypeptide comprising the amino acid sequence as set forth in SEQ ID NO:19 and further comprising the amino acid sequence as set forth in SEQ ID NO:6. In yet another embodiment, the IL-2 mutein comprises a first polypeptide comprising the amino acid sequence as set forth in SEQ ID NO:19 and further comprising the amino acid sequence as set forth in SEQ ID NO:7.

In other embodiments of the IL-2 muteins of the invention, the first polypeptide further comprises a linker which comprises the amino acid sequence as set forth in SEQ ID NO:8, 15, 16, 23 or 24. In one embodiment, the first polypeptide further comprises a linker as set forth in SEQ ID NO: 8, 15, 16, 23 or 24. In one embodiment, the IL-2 mutein comprises a first polypeptide comprising the amino acid sequence as set forth in SEQ ID NO: 3, 4, 18, or 19 and further comprising the amino acid sequence as set forth in SEQ ID NO:5, 6, or 7 and a linker as set forth in SEQ ID NO: 8, 15, 16, 23 or 24.

In other embodiments of the IL-2 muteins of the invention, the first polypeptide further comprises a linker which comprises the amino acid sequence as set forth in SEQ ID NO: 8, 15, or 16. In one embodiment, the first polypeptide further comprises a linker as set forth in SEQ ID NO: 8, 15, or 16. In one embodiment, the IL-2 mutein comprises a first polypeptide comprising the amino acid sequence as set forth in SEQ ID NO: 3, 4, 18, or 19 and further comprising the amino acid sequence as set forth in SEQ ID NO:5, 6, or 7 and a linker as set forth in SEQ ID NO:8, 15, or 16.

In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:8. In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:15. In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:16. In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:23. In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:24.

In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:8. In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:15. In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:16. In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:23. In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:24.

In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO:8. In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO:15. In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO:16. In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO: 23. In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO:24.

In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:8. In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:15. In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:16. In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:23. In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:24.

In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:8. In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:15. In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:16. In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:23. In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:24.

In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:8. In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:15. In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:16. In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:23. In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:24.

In one embodiment, the IL-2 mutein comprises a first polypeptide comprising the amino acid sequence as set forth in SEQ ID NO: 18 and further comprising the amino acid sequence as set forth in SEQ ID NO:5 and a linker as set forth in SEQ ID NO:8. In one embodiment, the IL-2 mutein comprises a first polypeptide comprising the amino acid sequence as set forth in SEQ ID NO:18 and further comprising the amino acid sequence as set forth in SEQ ID NO:5 and a linker as set forth in SEQ ID NO:15. In one embodiment, the IL-2 mutein comprises a first polypeptide comprising the amino acid sequence as set forth in SEQ ID NO:18 and further comprising the amino acid sequence as set forth in SEQ ID NO:5 and a linker as set forth in SEQ ID NO:16. In one embodiment, the IL-2 mutein comprises a first polypeptide comprising the amino acid sequence as set forth in SEQ ID NO:18 and further comprising the amino acid sequence as set forth in SEQ ID NO:5 and a linker as set forth in SEQ ID NO:23. In one embodiment, the IL-2 mutein comprises a first polypeptide comprising the amino acid sequence as set forth in SEQ ID NO:18 and further comprising the amino acid sequence as set forth in SEQ ID NO:5 and a linker as set forth in SEQ ID NO:24.

In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:8. In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:15. In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:16. In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:23. In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:24.

In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:8. In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:15. In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:16. In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:23. In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:24.

In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:8. In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:15. In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:16. In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:23. In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:24.

In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:8. In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:15. In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:16. In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:23. In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:24.

In one embodiment, the IL-2 mutein comprises a first polypeptide comprising the amino acid sequence as set forth in SEQ ID NO:19 and further comprising the amino acid sequence as set forth in SEQ ID NO:7 and a linker as set forth in SEQ ID NO:8. In one embodiment, the IL-2 mutein comprises a first polypeptide comprising the amino acid sequence as set forth in SEQ ID NO:19 and further comprising the amino acid sequence as set forth in SEQ ID NO:7 and a linker as set forth in SEQ ID NO:15. In one embodiment, the IL-2 mutein comprises a first polypeptide comprising the amino acid sequence as set forth in SEQ ID NO:19 and further comprising the amino acid sequence as set forth in SEQ ID NO:7 and a linker as set forth in SEQ ID NO:16. In one embodiment, the IL-2 mutein comprises a first polypeptide comprising the amino acid sequence as set forth in SEQ ID NO:19 and further comprising the amino acid sequence as set forth in SEQ ID NO:7 and a linker as set forth in SEQ ID NO:23. In one embodiment, the IL-2 mutein comprises a first polypeptide comprising the amino acid sequence as set forth in SEQ ID NO:19 and further comprising the amino acid sequence as set forth in SEQ ID NO:7 and a linker as set forth in SEQ ID NO:24.

In various embodiments, the IL-2 mutein further comprises a second polypeptide.

In one embodiment, the IL-2 mutein comprises a first polypeptide comprising the amino acid sequence as set forth in SEQ ID NO:3, 4, 18, or 19 and the amino acid sequence as set forth in SEQ ID NO:6 and a second polypeptide comprising the amino acid sequence as set forth in SEQ ID NO:10. In one embodiment, the IL-2 mutein comprises a first polypeptide comprising the amino acid sequence as set forth in SEQ ID NO:3 and the amino acid sequence as set forth in SEQ ID NO:6 and a second polypeptide comprising the amino acid sequence as set forth in SEQ ID NO:10. In one embodiment, the IL-2 mutein comprises a first polypeptide comprising the amino acid sequence as set forth in SEQ ID NO:4 and the amino acid sequence as set forth in SEQ ID NO:6 and a second polypeptide comprising the amino acid sequence as set forth in SEQ ID NO:10. In one embodiment, the IL-2 mutein comprises a first polypeptide comprising the amino acid sequence as set forth in SEQ ID NO:18 and the amino acid sequence as set forth in SEQ ID NO:6 and a second polypeptide comprising the amino acid sequence as set forth in SEQ ID NO:10. In one embodiment, the IL-2 mutein comprises a first polypeptide comprising the amino acid sequence as set forth in SEQ ID NO:19 and the amino acid sequence as set forth in SEQ ID NO:6 and a second polypeptide comprising the amino acid sequence as set forth in SEQ ID NO:10.

In another embodiment, the IL-2 mutein comprises a first polypeptide comprising the amino acid sequence as set forth in SEQ ID NO:3, 4, 18, or 19 and the amino acid sequence as set forth in SEQ ID NO:7 and a second polypeptide comprising the amino acid sequence as set forth in SEQ ID NO:9. In one embodiment, the IL-2 mutein comprises a first polypeptide comprising the amino acid sequence as set forth in SEQ ID NO:3 and the amino acid sequence as set forth in SEQ ID NO:7 and a second polypeptide comprising the amino acid sequence as set forth in SEQ ID NO:9. In one embodiment, the IL-2 mutein comprises a first polypeptide comprising the amino acid sequence as set forth in SEQ ID NO:4 and the amino acid sequence as set forth in SEQ ID NO:7 and a second polypeptide comprising the amino acid sequence as set forth in SEQ ID NO:9. In one embodiment, the IL-2 mutein comprises a first polypeptide comprising the amino acid sequence as set forth in SEQ ID NO:18 and the amino acid sequence as set forth in SEQ ID NO:7 and a second polypeptide comprising the amino acid sequence as set forth in SEQ ID NO:9. In one embodiment, the IL-2 mutein comprises a first polypeptide comprising the amino acid sequence as set forth in SEQ ID NO:19 and the amino acid sequence as set forth in SEQ ID NO:7 and a second polypeptide comprising the amino acid sequence as set forth in SEQ ID NO:9.

In one embodiment, the IL-2 mutein comprises a first polypeptide comprising the amino acid sequence as set forth in SEQ ID NO:3, 4, 18, or 19 and the amino acid sequence as set forth in SEQ ID NO:5 and a second polypeptide comprising the same amino acid sequence as the first polypeptide. In one embodiment, the IL-2 mutein comprises a first polypeptide comprising the amino acid sequence as set forth in SEQ ID NO:3 and the amino acid sequence as set forth in SEQ ID NO:5 and a second polypeptide comprising the same amino acid sequence as the first polypeptide. In one embodiment, the IL-2 mutein comprises a first polypeptide comprising the amino acid sequence as set forth in SEQ ID NO:4 and the amino acid sequence as set forth in SEQ ID NO:5 and a second polypeptide comprising the same amino acid sequence as the first polypeptide. In one embodiment, the IL-2 mutein comprises a first polypeptide comprising the amino acid sequence as set forth in SEQ ID NO:18 and the amino acid sequence as set forth in SEQ ID NO:5 and a second polypeptide comprising the same amino acid sequence as the first polypeptide. In one embodiment, the IL-2 mutein comprises a first polypeptide comprising the amino acid sequence as set forth in SEQ ID NO:19 and the amino acid sequence as set forth in SEQ ID NO:5 and a second polypeptide comprising the same amino acid sequence as the first polypeptide.

In one embodiment, the IL-2 mutein comprises a first polypeptide and a second polypeptide, wherein the first polypeptide comprises the amino acid sequence as set forth in SEQ ID NO:11; and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO:12; and the second polypeptide comprises the amino acid sequence as set forth in SEQ ID NO:9. In one embodiment, the IL-2 mutein comprises a first polypeptide and a second polypeptide, wherein the first polypeptide and the second polypeptide both comprise the amino acid sequence as set forth in SEQ ID NO:17. In one embodiment, the IL-2 mutein comprises a first polypeptide and a second polypeptide, wherein the first polypeptide comprises the amino acid sequence as set forth in SEQ ID NO:20; and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO:21; and the second polypeptide comprises the amino acid sequence as set forth in SEQ ID NO:9. In one embodiment, the IL-2 mutein comprises a first polypeptide and a second polypeptide, wherein the first polypeptide and the second polypeptide both comprise the amino acid sequence as set forth in SEQ ID NO:22.

In one embodiment, the IL-2 mutein comprises a first polypeptide and a second polypeptide, wherein the first polypeptide comprises the amino acid sequence as set forth in SEQ ID NO: 25 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 26 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 27 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 28 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 29 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 30 and the second polypeptide comprises the amino acid sequence as set forth in SEQ ID NO: 9. In one embodiment, the IL-2 mutein comprises a first polypeptide and a second polypeptide, wherein the first polypeptide comprises the amino acid sequence as set forth in SEQ ID NO: 31 and the second polypeptide comprises the amino acid sequence as set forth in SEQ ID NO:9.

In one embodiment, the IL-2 mutein consists of a first polypeptide and a second polypeptide, wherein the first polypeptide consists of the amino acid sequence as set forth in SEQ ID NO:11; and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO:12; and the second polypeptide consists of the amino acid sequence as set forth in SEQ ID NO:9. In one embodiment, the IL-2 mutein consists of a first polypeptide and a second polypeptide, wherein the first polypeptide and the second polypeptide both consist of the amino acid sequence as set forth in SEQ ID NO:17. In one embodiment, the IL-2 mutein consists of a first polypeptide and a second polypeptide, wherein the first polypeptide consists of the amino acid sequence as set forth in SEQ ID NO:21; and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO:20; and the second polypeptide consists of the amino acid sequence as set forth in SEQ ID NO:9. In one embodiment, the IL-2 mutein consists of a first polypeptide and a second polypeptide, wherein the first polypeptide and the second polypeptide both consist of the amino acid sequence as set forth in SEQ ID NO:22.

In one embodiment, the IL-2 mutein consists of a first polypeptide and a second polypeptide, wherein the first polypeptide consists of the amino acid sequence as set forth in SEQ ID NO: 25 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 26 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 27 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 28 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 29 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 30 and the second polypeptide consists of the amino acid sequence as set forth in SEQ ID NO: 9. In one embodiment, the IL-2 mutein consists of a first polypeptide and a second polypeptide, wherein the first polypeptide consists of the amino acid sequence as set forth in SEQ ID NO: 31 and the second polypeptide consists of the amino acid sequence as set forth in SEQ ID NO:9.

In another aspect, provided is a pharmaceutical composition comprising one or more of the various IL-2 muteins described herein and a pharmaceutically acceptable carrier.

In yet another aspect, provided is a method of treating an IL-2-mediated disease in a subject, comprising administering to the subject a therapeutically effective amount of one or more of the various IL-2 mutein described herein or the various pharmaceutical composition described herein.

In certain embodiments, the IL-2-mediated disease is cancer. In other embodiments, the IL-2-mediated disease is infection.

In some embodiments, the cancer is osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, or carcinoid cancer.

In certain embodiments, the cancer is metastatic. In some embodiments, the cancer is relapsed. In other embodiments, the cancer is refractory. In yet other embodiments, the cancer is relapsed and refractory. In some embodiments, the cancer is first-line. In other embodiments, the cancer is second line or third line.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelofibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer.

In still another aspect, provided is a method of selectively activating CD8+ T cells or NK cells without significantly activating T regulatory cells in a subject in need thereof, comprising administering to the subject a therapeutically effective amount one or more of the various IL-2 muteins described herein or one or more the various pharmaceutical compositions described herein.

In yet still another aspect, provided is a method of selectively activating cells that express IL-2 receptor βγ subunits but without significantly activating cells that express IL-2 receptor αβγ subunits compared to wild type IL-2 in a subject in need thereof, comprising administering to the subject a therapeutically effective amount of one or more of the various IL-2 muteins described herein or one or more of the various pharmaceutical compositions described herein.

In one aspect, provided is an isolated nucleic acid, encoding any one of the various polypeptides of the various IL-2 muteins described herein. In one embodiment, the isolated nucleic acid encodes one of the various first polypeptides of the various IL-2 muteins described herein. In another embodiment, the isolated nucleic acid encodes one of the various second polypeptides of the various IL-2 muteins described herein. In yet another embodiment, the isolated nucleic acid encodes one of the various first polypeptides and one of the various second polypeptides of the various IL-2 muteins described herein.

In another aspect, provided is an expression vector comprising any one of the various isolated nucleic acids described herein.

In yet another aspect, provided is a host cell comprising one of the various isolated nucleic acids described herein or one of the various expression vectors described herein.

In still another aspect, provided is a method of producing any one of the various IL-2 muteins described herein. In one embodiment, the method comprises culturing a host cell as described in any embodiment herein under conditions wherein the IL-2 mutein is expressed. In another embodiment, the method comprises expressing an expression vector as described in any embodiment herein under conditions wherein the IL-2 mutein is expressed. In yet another embodiment, the method comprises expressing any one of the various isolated nucleic acids described herein under conditions wherein the IL-2 mutein is expressed.

In yet still another aspect, provided is use of any one of the various IL-2 muteins described herein or any one of the various pharmaceutical compositions described herein to treat an IL-2-mediated disease in a subject.

In one aspect, provided is the use of any one of the various IL-2 muteins described herein or any one of the various pharmaceutical compositions described herein for the treatment of an IL-2-mediated disease in a subject.

In another aspect, provided is use of any one of the various IL-2 muteins described herein or any one of the various pharmaceutical compositions described herein for the preparation of a medicament to treat an IL-2-mediated disease in a subject.

In some embodiments of the various uses described herein, the IL-2-mediated disease is cancer. In other embodiments of the various uses described herein, the IL-2-mediated disease is infection.

In some embodiments, the cancer is osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, or carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelofibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer.

In one embodiment, the cancer is advanced renal cell carcinoma. In another embodiment, the cancer is metastatic melanoma.

In certain embodiments of the methods or uses described herein, the IL-2 is administered or used in combination with a second agent. In some embodiments, the second agent is a PD-1 antagonist, a PD-L1 antagonist, a CTLA4 antagonist, a LAG3 antagonist, a TIGIT antagonist, an ILT3 antagonist, an ILT4 antagonist, a PARP antagonist, a VEGF receptor antagonist, a FGF receptor antagonist, a HIF-2a antagonist, a BTK antagonist, an AKT antagonist, an ERK antagonist, a MEK antagonist, a CD27 agonist, a STING agonist, a chemotherapeutic agent, or an antibody-drug conjugate.

In one embodiment, the second agent is a PD-1 antagonist. In some embodiments, the PD-1 antagonist is pembrolizumab. In certain embodiments, the PD-1 antagonist is nivolumab. In other embodiments, the PD-1 antagonist is cemiplimab. In yet other embodiments, the PD-1 antagonist is pidilizumab. In other embodiments, the PD-1 antagonist is dostarlimab.

In another embodiment, the second agent is a PD-L1 antagonist. In some embodiments, the PD-L1 antagonist is atezolizumab. In certain embodiments, the PD-L1 antagonist is durvalumab. In other embodiments, the PD-L1 antagonist is avelumab.

In one embodiment, the second agent is a CTLA4 antagonist. In another embodiment, the second agent is a LAG3 antagonist. In yet another embodiment, the second agent is a TIGIT antagonist. In still another embodiment, the second agent is an ILT3 antagonist. In yet still another embodiment, the second agent is an ILT4 antagonist. In one embodiment, the second agent is a PARP antagonist. In another embodiment, the second agent is a VEGF receptor antagonist. In yet another embodiment, the second agent is a FGF receptor antagonist. In still another embodiment, the second agent is a HIF-2α antagonist. In yet still another embodiment, the second agent is a BTK antagonist. In one embodiment, the second agent is an AKT antagonist. In another embodiment, the second agent is an ERK antagonist. In yet another embodiment, the second agent is a MEK antagonist. In still another embodiment, the second agent is a CD27 agonist. In yet still another embodiment, the second agent is a STING agonist. In one embodiment, the second agent is a chemotherapeutic agent. In another embodiment, the second agent is an antibody-drug conjugate.

In certain embodiments, the IL-2 mutein and the second agent are administered or used simultaneously. In some embodiments, the IL-2 mutein and the second agent are administered or used sequentially. In certain embodiments, the IL-2 mutein and the second agent are administered or used through the same routes. In some embodiments, the IL-2 mutein and the second agent are administered or used through different routes.

### BRIEF DESCRIPTION OF THE DRAWINGS

FIGS. 1A and 1B show the effect of aldesleukin, WT-IL-2 Fc, or Y45R-IL-2 Fc on U2OS-IL-2Rβγ (FIG. 1A) or U2OS-IL-2Rαβ (FIG. 1B) dimerization after a 6-hour treatment.

FIG. 2 shows the effect of aldesleukin, WT-IL-2 Fc, or Y45R-IL-2 Fc on STAT5 phosphorylation in CTLL-2 cells.

FIGS. 3A-3F show that WT-IL-2 Fc and Y45R-IL-2 Fc elicited STAT5 phosphorylation in rhesus Treg (FIG. 3A), CD8+T cells (FIG. 3B), or NK cells (FIG. 3C) and human Treg cells (FIG. 3D), CD8+ T cells (FIG. 3E), or NK cells (FIG. 3F) in in vitro assays.

FIGS. 4A-4F show that aldesleukin elicited STAT5 phosphorylation in naïve C57BL/6 mice CD8+ T cells (FIG. 4A), NK cells (FIG. 4B), or CD4+ T cells (FIG. 4C) and Y45R-IL-2 Fc elicited STAT5 phosphorylation in naïve mice CD8+ T cells (FIG. 4D), NK cells (FIG. 4E), or CD4+ T cells (FIG. 4F).

FIGS. 5A-5H show the effect of aldesleukin on cell expansion in naïve C57BL/6 mice CD8+ T cells (FIG. 5A), NK cells (FIG. 5B), Treg cells (FIG. 5C), or CD4+ T cells (FIG. 5D) and Y45R-IL-2 Fc on cell expansion in naïve mice CD8+ T cells (FIG. 5E), NK cells (FIG. 5F), Treg cells (FIG. 5G), or CD4+ T cells (FIG. 5H).

FIGS. 6A and 6B show the effect of Y45R-IL-2 Fc (FIG. 6A) or WT-IL-2 Fc (FIG. 6B) on pSTAT5 in rhesus CD8+ T, NK and Treg cell populations.

FIGS. 7A-7H show the effect of WT-IL-2 Fc or Y45R-IL-2 Fc on tumor size (FIG. 7A) and different cell populations in the tumor microenvironment of B16F10 mice: percentage of CD45+ cells in live cells (FIG. 7B), ratio of CD8+ T cells to CD4+ T cells (FIG. 7C), percentage of CD8+ T cells in CD45+ cells (FIG. 7D), percentage of NK cells in CD45+ cells (FIG. 7E), percentage of GranzymeB+ cells in CD8+ T cells and NK cells (FIG. 7F), percentage of macrophages in CD45+ cells (FIG. 7G), percentage of monocytes and dendritic cells in CD45+ cells (FIG. 7H).

FIGS. 8A-8G show the effect of WT-IL-2 Fc or Y45R-IL-2 Fc on spleen weights (FIG. 8G) and different cells populations in the periphery of B16F10 mice: percentage of CD8+ T cells in CD45+ cells (FIG. 8A), percentage of GranzymeB+ cells in CD8+ T cells (FIG. 8B), percentage of GranzymeB+ cells in NK cells (FIG. 8C), percentage of Treg in CD45+ cells (FIG. 8D), percentage of dendritic cells in CD45+ cells (FIG. 8E), percentage of monocytes in CD45+ cells (FIG. 8F).

FIGS. 9A and 9B show the effect of Y45R-IL-2 Fc alone or in combination with DX400 on tumor volume (FIG. 9A) and body weight (FIG. 9B) in the CT26 mouse syngeneic tumor model.

### DETAILED DESCRIPTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise. Similarly, the plural forms of words include their corresponding singular references unless the context clearly indicates otherwise.

“IL-2 mutein” refers to a molecule comprising a partial or full-length human IL-2 amino acid sequence with one or more amino acid substitutions, deletions, or additions relative to the wild-type IL-2 amino acid sequence. The term “IL-2 mutein” includes any fusion proteins, protein conjugates, or multi subunit proteins (e.g., dimeric) that comprise a partial or full-length human IL-2 amino acid sequence with one or more amino acid substitutions, deletions, or additions. In certain embodiments, the IL-2 mutein comprises one partial or full-length human IL-2 amino acid sequence with one or more amino acid substitutions, deletions, or additions and thus is referred to as “monovalent” IL-2 mutein. In some embodiments, the IL-2 mutein comprises two partial or full-length human IL-2 amino acid sequences, each with one or more amino acid substitutions, deletions, or additions, and thus is referred to as “bivalent” IL-2 mutein. The full-length wild type human IL-2 amino acid sequence is set forth in SEQ ID NO:13. The partial human IL-2 amino acid sequence comprises at least 50%, 60%, 70%, 80%. 90%, 95%, 96%, 97%, 98%, or 99% of the full-length wild type human IL-2 amino acid sequence as set forth in SEQ ID NO:13.

As used herein, each specific IL-2 mutein, such as “T3A,” “C125S,” “Y45R,” or “R38Q/Y45F,” encompasses a partial or full-length human IL-2 amino acid sequence with its specific amino acid substitution(s) as indicated, wherein the position of the amino acid substitution(s) is relevant to the human wild type IL-2 amino acid sequence as set forth in SEQ ID NO:13. Each IL-2 mutein can be used in the form of a fusion protein (e.g., Fc fusion), protein conjugate, or multi subunit protein (e.g., dimeric).

A “Fc” region or domain refers to the heavy chain fragment comprising the CH2 and CH3 domains of an antibody. The antibody can be of any class (e.g., IgG, IgE, IgM, IgD, and IgA) or any subclass (e.g., IgG1, IgG2, IgG3, IgG4, IgA1, and IgA2) of immunoglobulin molecule. Two Fc regions or domains can form a dimer by two or more disulfide bonds and by hydrophobic interactions of the CH3 domains. A “Fc variant” contains one or more amino acid substitutions, deletions, or insertions compared to a wild type Fc region or domain.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. Unless indicated otherwise, as used herein, “binding affinity” refers to intrinsic binding affinity which reflects a 1:1 interaction between members of a binding pair (e.g., an IL-2 mutein and an IL-2 receptor subunit). The ratio of dissociation rate (k_{off}) to association rate (kₒₙ) of an IL-2 mutein to an IL-2 receptor subunit (k_{off}/kₒₙ) is the “dissociation constant” (or “equilibrium dissociation constant” as used interchangeably) KD, which is inversely related to affinity. The lower the KD value, the higher the affinity. The value of KD varies for different complexes of IL-2 mutein and IL-2 receptor subunit and depends on both kₒₙ and k_{off}. The dissociation constant KD for an IL-2 mutein provided herein can be determined using any method provided herein or any other method well known to those skilled in the art, such as surface plasmon resonance (SPR) assay, including but not limited to Biacore and KinExA.

“Administrating” or “administration,” as it applies to an animal, human, subject, cell, tissue, organ, or biological fluid, refers to contact of an exogenous pharmaceutical, therapeutic, diagnostic agent, or composition to the animal, human, subject, cell, tissue, organ, or biological fluid.

The term “subject” includes any organism, preferably an animal, more preferably a mammal (e.g., human, rat, mouse, dog, cat, or rabbit). In a preferred embodiment, the term “subject” refers to a human.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

As used herein, the expressions “cell,” “host cell,” “cell line,” and “cell culture” are used interchangeably and all such designations include progeny.

“Treat” or “treatment” means to administer an agent, such as a composition containing any of the IL-2 muteins of the present invention, internally or externally to a subject or patient having one or more disease symptoms, or being suspected of having a disease, for which the agent has therapeutic activity. A subject “in need of treatment” includes those subjects having an IL-2 mediated disease or disorder, those at risk of developing such a disease or disorder and those suspected of having such disease or disorder. Typically, the agent is administered in an amount effective to alleviate one or more disease symptoms in the treated subject or population, whether by inducing the regression of or inhibiting, delaying or slowing the progression of such symptom(s) by any clinically measurable degree. The amount of an agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

The terms “prevent.” “preventing.” and “prevention” refer to reducing the likelihood of the onset (or recurrence) of a disease, disorder, condition, or associated symptom(s) (e.g., cancer).

An “IL-2-mediated disease,” “IL-2-mediated disorder,” and “IL-2-mediated condition” are used interchangeably and refer to any disease, disorder, or condition that is completely or partially caused by or is the result of IL-2 signaling and/or alternatively any disease, disorder, or condition in which it is desirable to modulate IL-2 signaling, either systematically or in selected cell types, tissues, or organs.

The term “therapeutically effective amount” as used herein refers to the amount of an agent (e.g., an IL-2 mutein provided herein or any other agent described herein) that is sufficient to reduce and/or ameliorate the severity and/or duration of a given disease, disorder, or condition, and/or a symptom related thereto (e.g., cancer). A “therapeutically effective amount” of a substance/molecule/agent of the present disclosure (e.g., an IL-2 mutein) may vary according to factors such as the disease state, age, sex, and weight of the individual, and the ability of the substance/molecule/agent to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of an IL-2 mutein or other agent (e.g., drug) effective to “treat” a disease, disorder, or condition, in a subject or mammal.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, may contain an IL-2 mutein, for example, in isolated or purified form, together with a suitable number of carriers.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

An “isolated nucleic acid” is a nucleic acid, for example, an RNA, DNA, or a mixed nucleic acid, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an IL-2 mutein as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an IL-2 mutein of the present disclosure may include a host cell into which nucleic acids encoding the IL-2 mutein have been introduced. Suitable host cells are disclosed below.

“PD-1 antagonist” means any chemical compound or biological molecule that binds to PD-1 and blocks binding of PD-1 to PD-L1 and preferably also blocks binding of PD-1 to PD-L2. “PD-L1 antagonist” means any chemical compound or biological molecule that binds to PD-L1 and blocks binding of PD-L1 to PD-1 but does not block binding of PD-L2 to PD-1. Alternative names or synonyms for PD-1 and its ligands include: PDCD1, PD1, CD279 and SLEB2 for PD-1; PDCDIL1, PDL1, B7H1, B7-4, CD274 and B7-H for PD-L1; and PDCDIL2, PDL2, B7-DC, Btdc and CD273 for PD-L2. Human PD-1 amino acid sequences can be found in NCBI Locus No.: NP_005009. Human PD-L1 and PD-L2 amino acid sequences can be found in NCBI Locus No.: NP_054862 and NP_079515, respectively.

The term “infection” refers to any infectious diseases, including but not limited to, bacterial infection, viral infection, or fungal infection.

### IL-2 Muteins

This disclosure provides IL-2 muteins that bind to the IL-2 receptor β subunit but do not have measurable binding to the IL-2 receptor a subunit.

The various IL-2 muteins described herein can be a partial or full-length human IL-2 molecule comprising one or more amino acid substitutions, deletions, or additions relative to the wild-type IL-2 amino acid sequence. In some embodiments, the partial or full-length human IL-2 molecule comprising one or more substitutions, deletions, or additions is fused to another polypeptide, such as a Fc region of a human immunoglobulin (e.g., IgG1, IgG2, IgG3, or IgG4). The Fc region can be a wild type Fc or a Fc variant with desired characteristics or properties, such as modified serum half-life, complement fixation, Fc receptor binding, and/or effector function (e.g., antigen-dependent cellular cytotoxicity).

In one embodiment, the Fc region is modified with two amino acid substitutions, L234A and L235A (sometimes referred to as “LALA” mutations) that reduce/ablate effector function. In another embodiment, the Fc region is modified with three amino acid substitutions, L234A, L235A and D265S that reduce/ablate effector function (sometimes referred to as “LALADS” mutations). In yet another embodiment, the Fc region is modified with three amino acid substitutions, M252Y, S254T, and T256E that increase half-life in serum (sometimes referred to as “YTE” mutations). In still another embodiment, the Fc variant has a combination of different mutations described herein, for example, LALA and YTE mutations, or LALADS and YTE mutations. The partial or full-length human IL-2 molecule comprising one or more substitutions, deletions, or additions fused with a Fc variant can form homodimers through the Fc variant. Such homodimers contain two IL-2 mutant molecules and are thus bivalent IL-2 muteins.

In other embodiments, a pair of Fc variants include one Fc region comprising a knob mutation (“Fc knob”) and another Fc region comprising a hole mutation (“Fc hole”). The Fc knob and the Fc hole can form a heterodimer (sometimes referred to as “knob in hole” mutations). In certain embodiments, the Fc region is modified with two amino acid substitutions, S354C and T366W (sometimes referred to as “knob” mutations). In other embodiments, the Fc region is modified with four amino acid substitutions, Y349C, T366S, L368A, and Y407V (sometimes referred to as “hole” mutations). The partial or full-length human IL-2 molecule comprising one or more substitutions, deletions, or additions can be fused with Fc knob or Fc hole. In some embodiments, the partial or full-length human IL-2 molecule comprising one or more substitutions, deletions, or additions fused with Fc knob is paired with Fc hole to form a monovalent IL-2 mutein. In certain embodiments, the partial or full-length human IL-2 molecule comprising one or more substitutions, deletions, or additions fused with Fc hole is paired with Fc knob to form a monovalent IL-2 mutein. In other embodiments, the partial or full-length human IL-2 molecule comprising one or more substitutions, deletions, or additions is fused with Fc without either knob or hole, and the Fc-fused IL-2 molecule can form a homodimer bivalent IL-2 mutein.

The partial or full-length human IL-2 molecule comprising one or more substitutions, deletions, or additions can be fused to the N-terminus or C-terminus of a Fc variant, through a variety of peptide linkers between the IL-2 molecule and the Fc variant. In one embodiment, the partial or full-length human IL-2 molecule comprising one or more substitutions, deletions, or additions is fused to the N-terminus of a Fc variant, and the peptide linker is between the C-terminus of the IL-2 molecule and the N-terminus of the Fc variant. In another embodiment, the partial or full-length human IL-2 molecule comprising one or more substitutions, deletions, or additions is fused to the C-terminus of a Fc variant, and the peptide linker is between the C-terminus of the Fc variant and the N-terminus of the IL-2 molecule.

In certain embodiments, provided is an IL-2 mutein that comprises a first polypeptide comprising the amino acid sequence as set forth in SEQ ID NO:3, 4, 18, or 19. In one embodiment, the IL-2 mutein comprises a first polypeptide comprising the amino acid sequence as set forth in SEQ ID NO:3. In another embodiment, the IL-2 mutein comprises a first polypeptide comprising the amino acid sequence as set forth in SEQ ID NO:4. In one embodiment, the IL-2 mutein comprises a first polypeptide comprising the amino acid sequence as set forth in SEQ ID NO:18. In another embodiment, the IL-2 mutein comprises a first polypeptide comprising the amino acid sequence as set forth in SEQ ID NO:19.

In some embodiments of the IL-2 muteins, the first polypeptide further comprises the amino acid sequence as set forth in SEQ ID NO:5, 6, or 7. In one embodiment, the IL-2 mutein comprises a first polypeptide comprising the amino acid sequence as set forth in SEQ ID NO:3, 4, 18, or 19 and further comprising the amino acid sequence as set forth in SEQ ID NO:5, 6, or 7. In another embodiment, the IL-2 mutein comprises a first polypeptide comprising the amino acid sequence as set forth in SEQ ID NO:3 and further comprising the amino acid sequence as set forth in SEQ ID NO:5. In yet another embodiment, the IL-2 mutein comprises a first polypeptide comprising the amino acid sequence as set forth in SEQ ID NO:3 and further comprising the amino acid sequence as set forth in SEQ ID NO:6. In still another embodiment, the IL-2 mutein comprises a first polypeptide comprising the amino acid sequence as set forth in SEQ ID NO:3 and further comprising the amino acid sequence as set forth in SEQ ID NO:7. In yet still another embodiment, the IL-2 mutein comprises a first polypeptide comprising the amino acid sequence as set forth in SEQ ID NO:4 and further comprising the amino acid sequence as set forth in SEQ ID NO:5. In another embodiment, the IL-2 mutein comprises a first polypeptide comprising the amino acid sequence as set forth in SEQ ID NO:4 and further comprising the amino acid sequence as set forth in SEQ ID NO:6. In yet another embodiment, the IL-2 mutein comprises a first polypeptide comprising the amino acid sequence as set forth in SEQ ID NO:4 and further comprising the amino acid sequence as set forth in SEQ ID NO:7. In another embodiment, the IL-2 mutein comprises a first polypeptide comprising the amino acid sequence as set forth in SEQ ID NO:18 and further comprising the amino acid sequence as set forth in SEQ ID NO:5. In yet another embodiment, the IL-2 mutein comprises a first polypeptide comprising the amino acid sequence as set forth in SEQ ID NO:18 and further comprising the amino acid sequence as set forth in SEQ ID NO:6. In still another embodiment, the IL-2 mutein comprises a first polypeptide comprising the amino acid sequence as set forth in SEQ ID NO:18 and further comprising the amino acid sequence as set forth in SEQ ID NO:7. In yet still another embodiment, the IL-2 mutein comprises a first polypeptide comprising the amino acid sequence as set forth in SEQ ID NO:19 and further comprising the amino acid sequence as set forth in SEQ ID NO:5. In another embodiment, the IL-2 mutein comprises a first polypeptide comprising the amino acid sequence as set forth in SEQ ID NO:19 and further comprising the amino acid sequence as set forth in SEQ ID NO:6. In yet another embodiment, the IL-2 mutein comprises a first polypeptide comprising the amino acid sequence as set forth in SEQ ID NO:19 and further comprising the amino acid sequence as set forth in SEQ ID NO:7.

In other embodiments of the IL-2 muteins, the first polypeptide further comprises a linker as set forth in SEQ ID NO:8, 15, 16, 23 or 24. In one embodiment, the IL-2 mutein comprises a first polypeptide comprising the amino acid sequence as set forth in SEQ ID NO:3, 4, 18, or 19 and further comprising the amino acid sequence as set forth in SEQ ID NO:5, 6, or 7 and a linker as set forth in SEQ ID NO:8, 15, 16, 23 or 24.

In other embodiments of the IL-2 muteins, the first polypeptide further comprises a linker as set forth in SEQ ID NO:8, 15, or 16. In one embodiment, the IL-2 mutein comprises a first polypeptide comprising the amino acid sequence as set forth in SEQ ID NO:3, 4, 18, or 19 and further comprising the amino acid sequence as set forth in SEQ ID NO:5, 6, or 7 and a linker as set forth in SEQ ID NO:8, 15, or 16.

In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:8. In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:15. In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:16. In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:23. In one embodiment, the IL-2 mutein comprises a first polypeptide comprising the amino acid sequence as set forth in SEQ ID NO:3 and further comprising the amino acid sequence as set forth in SEQ ID NO:5 and a linker as set forth in SEQ ID NO:24.

In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:8. In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:15. In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:16. In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:23. In one embodiment, the IL-2 mutein comprises a first polypeptide comprising the amino acid sequence as set forth in SEQ ID NO:3 and further comprising the amino acid sequence as set forth in SEQ ID NO:6 and a linker as set forth in SEQ ID NO:24.

In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO:8. In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO:15. In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO:16. In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO:23. In one embodiment, the IL-2 mutein comprises a first polypeptide comprising the amino acid sequence as set forth in SEQ ID NO:3 and further comprising the amino acid sequence as set forth in SEQ ID NO:7 and a linker as set forth in SEQ ID NO:24.

In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:8. In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:15. In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:16. In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:23. In one embodiment, the IL-2 mutein comprises a first polypeptide comprising the amino acid sequence as set forth in SEQ ID NO:4 and further comprising the amino acid sequence as set forth in SEQ ID NO:5 and a linker as set forth in SEQ ID NO:24.

In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:8. In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:15. In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:16. In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:23. In one embodiment, the IL-2 mutein comprises a first polypeptide comprising the amino acid sequence as set forth in SEQ ID NO:4 and further comprising the amino acid sequence as set forth in SEQ ID NO:6 and a linker as set forth in SEQ ID NO:24.

In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:8. In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:15. In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:16. In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:23. In one embodiment, the IL-2 mutein comprises a first polypeptide comprising the amino acid sequence as set forth in SEQ ID NO:4 and further comprising the amino acid sequence as set forth in SEQ ID NO:7 and a linker as set forth in SEQ ID NO:24.

In one embodiment, the IL-2 mutein comprises a first polypeptide comprising the amino acid sequence as set forth in SEQ ID NO:18 and further comprising the amino acid sequence as set forth in SEQ ID NO:5 and a linker as set forth in SEQ ID NO:8. In one embodiment, the IL-2 mutein comprises a first polypeptide comprising the amino acid sequence as set forth in SEQ ID NO:18 and further comprising the amino acid sequence as set forth in SEQ ID NO:5 and a linker as set forth in SEQ ID NO:15. In one embodiment, the IL-2 mutein comprises a first polypeptide comprising the amino acid sequence as set forth in SEQ ID NO:18 and further comprising the amino acid sequence as set forth in SEQ ID NO:5 and a linker as set forth in SEQ ID NO:16. In one embodiment, the IL-2 mutein comprises a first polypeptide comprising the amino acid sequence as set forth in SEQ ID NO:18 and further comprising the amino acid sequence as set forth in SEQ ID NO:5 and a linker as set forth in SEQ ID NO:23. In one embodiment, the IL-2 mutein comprises a first polypeptide comprising the amino acid sequence as set forth in SEQ ID NO:18 and further comprising the amino acid sequence as set forth in SEQ ID NO:5 and a linker as set forth in SEQ ID NO:24.

In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:8. In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:15. In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:16. In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:23. In one embodiment, the IL-2 mutein comprises a first polypeptide comprising the amino acid sequence as set forth in SEQ ID NO:18 and further comprising the amino acid sequence as set forth in SEQ ID NO:6 and a linker as set forth in SEQ ID NO:24.

In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:8. In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:15. In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:16. In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:23. In one embodiment, the IL-2 mutein comprises a first polypeptide comprising the amino acid sequence as set forth in SEQ ID NO:18 and further comprising the amino acid sequence as set forth in SEQ ID NO:7 and a linker as set forth in SEQ ID NO:24.

In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:8. In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:15. In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:16. In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:23. In one embodiment, the IL-2 mutein comprises a first polypeptide comprising the amino acid sequence as set forth in SEQ ID NO:19 and further comprising the amino acid sequence as set forth in SEQ ID NO:5 and a linker as set forth in SEQ ID NO:24.

In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:8. In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:15. In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:16. In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:23. In one embodiment, the IL-2 mutein comprises a first polypeptide comprising the amino acid sequence as set forth in SEQ ID NO:19 and further comprising the amino acid sequence as set forth in SEQ ID NO:6 and a linker as set forth in SEQ ID NO:24.

In one embodiment, the IL-2 mutein comprises a first polypeptide comprising the amino acid sequence as set forth in SEQ ID NO:19 and further comprising the amino acid sequence as set forth in SEQ ID NO:7 and a linker as set forth in SEQ ID NO:8. In one embodiment, the IL-2 mutein comprises a first polypeptide comprising the amino acid sequence as set forth in SEQ ID NO: 19 and further comprising the amino acid sequence as set forth in SEQ ID NO:7 and a linker as set forth in SEQ ID NO:15. In one embodiment, the IL-2 mutein comprises a first polypeptide comprising the amino acid sequence as set forth in SEQ ID NO:19 and further comprising the amino acid sequence as set forth in SEQ ID NO:7 and a linker as set forth in SEQ ID NO:16. In one embodiment, the IL-2 mutein comprises a first polypeptide comprising the amino acid sequence as set forth in SEQ ID NO:19 and further comprising the amino acid sequence as set forth in SEQ ID NO:7 and a linker as set forth in SEQ ID NO:23. In one embodiment, the IL-2 mutein comprises a first polypeptide comprising the amino acid sequence as set forth in SEQ ID NO:19 and further comprising the amino acid sequence as set forth in SEQ ID NO:7 and a linker as set forth in SEQ ID NO:24.

In various embodiments, the IL-2 mutein further comprises a second polypeptide.

In one embodiment, the IL-2 mutein comprises a first polypeptide comprising the amino acid sequence as set forth in SEQ ID NO:3, 4, 18, or 19 and the amino acid sequence as set forth in SEQ ID NO:6 and a second polypeptide comprising the amino acid sequence as set forth in SEQ ID NO:10. In one embodiment, the IL-2 mutein comprises a first polypeptide comprising the amino acid sequence as set forth in SEQ ID NO:3 and the amino acid sequence as set forth in SEQ ID NO:6 and a second polypeptide comprising the amino acid sequence as set forth in SEQ ID NO:10. In one embodiment, the IL-2 mutein comprises a first polypeptide comprising the amino acid sequence as set forth in SEQ ID NO:4 and the amino acid sequence as set forth in SEQ ID NO:6 and a second polypeptide comprising the amino acid sequence as set forth in SEQ ID NO:10. In one embodiment, the IL-2 mutein comprises a first polypeptide comprising the amino acid sequence as set forth in SEQ ID NO:18 and the amino acid sequence as set forth in SEQ ID NO:6 and a second polypeptide comprising the amino acid sequence as set forth in SEQ ID NO:10. In one embodiment, the IL-2 mutein comprises a first polypeptide comprising the amino acid sequence as set forth in SEQ ID NO:19 and the amino acid sequence as set forth in SEQ ID NO:6 and a second polypeptide comprising the amino acid sequence as set forth in SEQ ID NO:10.

In another embodiment, the IL-2 mutein comprises a first polypeptide comprising the amino acid sequence as set forth in SEQ ID NO:3, 4, 18, or 19 and the amino acid sequence as set forth in SEQ ID NO:7 and a second polypeptide comprising the amino acid sequence as set forth in SEQ ID NO:9. In one embodiment, the IL-2 mutein comprises a first polypeptide comprising the amino acid sequence as set forth in SEQ ID NO:3 and the amino acid sequence as set forth in SEQ ID NO:7 and a second polypeptide comprising the amino acid sequence as set forth in SEQ ID NO:9. In one embodiment, the IL-2 mutein comprises a first polypeptide comprising the amino acid sequence as set forth in SEQ ID NO:4 and the amino acid sequence as set forth in SEQ ID NO:7 and a second polypeptide comprising the amino acid sequence as set forth in SEQ ID NO:9. In one embodiment, the IL-2 mutein comprises a first polypeptide comprising the amino acid sequence as set forth in SEQ ID NO:18 and the amino acid sequence as set forth in SEQ ID NO:7 and a second polypeptide comprising the amino acid sequence as set forth in SEQ ID NO:9. In one embodiment, the IL-2 mutein comprises a first polypeptide comprising the amino acid sequence as set forth in SEQ ID NO:19 and the amino acid sequence as set forth in SEQ ID NO:7 and a second polypeptide comprising the amino acid sequence as set forth in SEQ ID NO:9.

In one embodiment, the IL-2 mutein comprises a first polypeptide comprising the amino acid sequence as set forth in SEQ ID NO:3, 4, 18, or 19 and the amino acid sequence as set forth in SEQ ID NO:5 and a second polypeptide comprising the same amino acid sequence as the first polypeptide. In one embodiment, the IL-2 mutein comprises a first polypeptide comprising the amino acid sequence as set forth in SEQ ID NO:3 and the amino acid sequence as set forth in SEQ ID NO:5 and a second polypeptide comprising the same amino acid sequence as the first polypeptide. In one embodiment, the IL-2 mutein comprises a first polypeptide comprising the amino acid sequence as set forth in SEQ ID NO:4 and the amino acid sequence as set forth in SEQ ID NO:5 and a second polypeptide comprising the same amino acid sequence as the first polypeptide. In one embodiment, the IL-2 mutein comprises a first polypeptide comprising the amino acid sequence as set forth in SEQ ID NO:18 and the amino acid sequence as set forth in SEQ ID NO:5 and a second polypeptide comprising the same amino acid sequence as the first polypeptide. In one embodiment, the IL-2 mutein comprises a first polypeptide comprising the amino acid sequence as set forth in SEQ ID NO:19 and the amino acid sequence as set forth in SEQ ID NO:5 and a second polypeptide comprising the same amino acid sequence as the first polypeptide.

In one embodiment, the IL-2 mutein comprises a first polypeptide and a second polypeptide, wherein the first polypeptide comprises the amino acid sequence as set forth in SEQ ID NO:11; and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO:12; and the second polypeptide comprises the amino acid sequence as set forth in SEQ ID NO:9. In one embodiment, the IL-2 mutein comprises a first polypeptide and a second polypeptide, wherein the first polypeptide and the second polypeptide both comprise the amino acid sequence as set forth in SEQ ID NO:17. In one embodiment, the IL-2 mutein comprises a first polypeptide and a second polypeptide, wherein the first polypeptide comprises the amino acid sequence as set forth in SEQ ID NO:20; and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO:21; and the second polypeptide comprises the amino acid sequence as set forth in SEQ ID NO:9. In one embodiment, the IL-2 mutein comprises a first polypeptide and a second polypeptide, wherein the first polypeptide and the second polypeptide both comprise the amino acid sequence as set forth in SEQ ID NO:22.

In one embodiment, the IL-2 mutein comprises a first polypeptide and a second polypeptide, wherein the first polypeptide comprises the amino acid sequence as set forth in SEQ ID NO: 25 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 26 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 27 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 28 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 29 and the second polypeptide comprises the amino acid sequence as set forth in SEQ ID NO:10. In one embodiment, the IL-2 mutein comprises a first polypeptide and a second polypeptide, wherein the first polypeptide comprises the amino acid sequence as set forth in SEQ ID NO: 30 and the second polypeptide comprises the amino acid sequence as set forth in SEQ ID NO: 9. In one embodiment, the IL-2 mutein comprises a first polypeptide and a second polypeptide, wherein the first polypeptide comprises the amino acid sequence as set forth in SEQ ID NO: 31 and the second polypeptide comprises the amino acid sequence as set forth in SEQ ID NO:9.

In one embodiment, the IL-2 mutein consists of a first polypeptide and a second polypeptide, wherein the first polypeptide consists of the amino acid sequence as set forth in SEQ ID NO: 11; and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 12; and the second polypeptide consists of the amino acid sequence as set forth in SEQ ID NO:9. In one embodiment, the IL-2 mutein consists of a first polypeptide and a second polypeptide, wherein the first polypeptide and the second polypeptide both consist of the amino acid sequence as set forth in SEQ ID NO:17. In one embodiment, the IL-2 mutein consists of a first polypeptide and a second polypeptide, wherein the first polypeptide consists of the amino acid sequence as set forth in SEQ ID NO:21; and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO:20; and the second polypeptide consists of the amino acid sequence as set forth in SEQ ID NO:9. In one embodiment, the IL-2 mutein consists of a first polypeptide and a second polypeptide, wherein the first polypeptide and the second polypeptide both consist of the amino acid sequence as set forth in SEQ ID NO:22.

In one embodiment, the IL-2 mutein consists of a first polypeptide and a second polypeptide, wherein the first polypeptide consists of the amino acid sequence as set forth in SEQ ID NO: 25 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 26 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 27 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 28 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 29 and the second polypeptide consists of the amino acid sequence as set forth in SEQ ID NO:10. In one embodiment, the IL-2 mutein consists of a first polypeptide and a second polypeptide, wherein the first polypeptide consists of the amino acid sequence as set forth in SEQ ID NO: 30 and the second polypeptide consists of the amino acid sequence as set forth in SEQ ID NO: 9. In one embodiment, the IL-2 mutein consists of a first polypeptide and a second polypeptide, wherein the first polypeptide consists of the amino acid sequence as set forth in SEQ ID NO: 31 and the second polypeptide consists of the amino acid sequence as set forth in SEQ ID NO:9.

### Methods of Using IL-2 Muteins

The present disclosure also includes methods of using any one of the various IL-2 muteins described herein, including but not limited to preventing or treating an IL-2-mediated disease (e.g., cancer), selectively activating CD8+ T cells or NK cells without significantly activating T regulatory cells, or selectively activating cells that express IL-2 receptor βγ subunit but not significantly activating cells that express IL-2 receptor αβγ subunit compared to wild type IL-2.

Provided herein are methods of treating an IL-2-mediated disease in a subject. Also provided herein are methods of preventing an IL-2-mediated disease in a subject. In some embodiments, the subject has an IL-2-mediated disease. In other embodiments, the subject is at risk of having an IL-2-mediated disease.

In some embodiments of the various methods provided herein, the IL-2-mediated disease is cancer. In other embodiments, the IL-2-mediated disease is infection.

Thus, in some embodiments, provided is a method of treating an IL-2-mediated disease in a subject, comprising administering to the subject a therapeutically effective amount of any one of the various IL-2 muteins described herein or any one of the various pharmaceutical compositions described herein.

In certain embodiments, provided is a method of treating cancer in a subject, comprising administering to the subject a therapeutically effective amount of any one of the various IL-2 muteins described herein or any one of the various pharmaceutical compositions described herein.

In other embodiments, provided is a method of treating infection in a subject, comprising administering to the subject a therapeutically effective amount of any one of the various IL-2 muteins described herein or the any one of the various pharmaceutical compositions described herein.

In one embodiment, the subject was previously treated with a standard therapy, e.g. chemotherapy, prior to being treated with a therapeutically effective amount of an IL-2 mutein or pharmaceutical composition of the invention. In another embodiment, the subject was not previously treated with any other anti-cancer agent prior to treatment with an IL-2 mutein or pharmaceutical composition of the invention.

In some embodiments, the cancer is osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, or carcinoid cancer.

In one embodiment of any of the methods herein, the cancer is any solid tumor.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelofibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer.

In one embodiment, the cancer is advanced renal cell carcinoma. In another embodiment, the cancer is metastatic melanoma.

In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19 and the amino acid sequence as set forth in SEQ ID NO:6 and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19, the amino acid sequence as set forth in SEQ ID NO:6, and a linker sequence as set forth in SEQ ID NO:8, 15, or 16, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19, the amino acid sequence as set forth in SEQ ID NO:6, and a linker sequence as set forth in SEQ ID NO:8, 15, 16, 23 or 24, and a second polypeptide comprising the amino acid sequence as set forth in SEQ ID NO:10.

In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:6, and a linker sequence as set forth in SEQ ID NO:8, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:6, and a linker sequence as set forth in SEQ ID NO:15, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:6, and a linker sequence as set forth in SEQ ID NO:16, and a second polypeptide comprising the amino acid sequence as set forth in SEQ ID NO:10.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:6, and a linker sequence as set forth in SEQ ID NO:23, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:6, and a linker sequence as set forth in SEQ ID NO:24, and a second polypeptide comprising the amino acid sequence as set forth in SEQ ID NO:10.

In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:6, and a linker sequence as set forth in SEQ ID NO:8, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:6, and a linker sequence as set forth in SEQ ID NO:15, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:6, and a linker sequence as set forth in SEQ ID NO:16, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:6, and a linker sequence as set forth in SEQ ID NO:23, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:6, and a linker sequence as set forth in SEQ ID NO:24, and a second polypeptide comprising the amino acid sequence as set forth in SEQ ID NO:10.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:6, and a linker sequence as set forth in SEQ ID NO:8, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:6, and a linker sequence as set forth in SEQ ID NO:15, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:6, and a linker sequence as set forth in SEQ ID NO:16, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:6, and a linker sequence as set forth in SEQ ID NO:23, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:6, and a linker sequence as set forth in SEQ ID NO:24, and a second polypeptide comprising the amino acid sequence as set forth in SEQ ID NO:10.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:6, and a linker sequence as set forth in SEQ ID NO:8, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:6, and a linker sequence as set forth in SEQ ID NO:15, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:6, and a linker sequence as set forth in SEQ ID NO:16, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:6, and a linker sequence as set forth in SEQ ID NO:23, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:6, and a linker sequence as set forth in SEQ ID NO:24, and a second polypeptide comprising the amino acid sequence as set forth in SEQ ID NO:10.

In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19 and the amino acid sequence as set forth in SEQ ID NO:7 and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19, the amino acid sequence as set forth in SEQ ID NO:7, and a linker sequence as set forth in SEQ ID NO:8, 15, or 16, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19, the amino acid sequence as set forth in SEQ ID NO:7, and a linker sequence as set forth in SEQ ID NO:8, 15, 16, 23 or 24 and a second polypeptide comprising the amino acid sequence as set forth in SEQ ID NO:9.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:7, and a linker sequence as set forth in SEQ ID NO:8, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:7, and a linker sequence as set forth in SEQ ID NO:15, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:7, and a linker sequence as set forth in SEQ ID NO:16, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:7, and a linker sequence as set forth in SEQ ID NO:23, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:7, and a linker sequence as set forth in SEQ ID NO:24, and a second polypeptide comprising the amino acid sequence as set forth in SEQ ID NO:9.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:7, and a linker sequence as set forth in SEQ ID NO:8, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:7, and a linker sequence as set forth in SEQ ID NO:15, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:7, and a linker sequence as set forth in SEQ ID NO:16, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:7, and a linker sequence as set forth in SEQ ID NO:23, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:7, and a linker sequence as set forth in SEQ ID NO:24, and a second polypeptide comprising the amino acid sequence as set forth in SEQ ID NO:9.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:7, and a linker sequence as set forth in SEQ ID NO:8, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:7, and a linker sequence as set forth in SEQ ID NO:15, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:7, and a linker sequence as set forth in SEQ ID NO:16, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:7, and a linker sequence as set forth in SEQ ID NO:23, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:7, and a linker sequence as set forth in SEQ ID NO:24, and a second polypeptide comprising the amino acid sequence as set forth in SEQ ID NO:9.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:7, and a linker sequence as set forth in SEQ ID NO:8, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:7, and a linker sequence as set forth in SEQ ID NO:15, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:7, and a linker sequence as set forth in SEQ ID NO:16, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:7, and a linker sequence as set forth in SEQ ID NO:23, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:7, and a linker sequence as set forth in SEQ ID NO:24, and a second polypeptide comprising the amino acid sequence as set forth in SEQ ID NO:9.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19 and the amino acid sequence as set forth in SEQ ID NO:5 and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19, the amino acid sequence as set forth in SEQ ID NO:5, and a linker sequence as set forth in SEQ ID NO:8, 15, or 16, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19, the amino acid sequence as set forth in SEQ ID NO:5, and a linker sequence as set forth in SEQ ID NO:8, 15, or 23, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, 4, 18, or 19, the amino acid sequence as set forth in SEQ ID NO:5, and a linker sequence as set forth in SEQ ID NO:8, 15, or 24, and a second polypeptide comprising an amino acid sequence which is the same as the first polypeptide.

In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:5, and a linker sequence as set forth in SEQ ID NO:8, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:5, and a linker sequence as set forth in SEQ ID NO:15, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:5, and a linker sequence as set forth in SEQ ID NO:16, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:5, and a linker sequence as set forth in SEQ ID NO:23, and a second polypeptide comprising an amino acid sequence the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:3, the amino acid sequence as set forth in SEQ ID NO:5, and a linker sequence as set forth in SEQ ID NO:24, and a second polypeptide comprising an amino acid sequence the same as the first polypeptide.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:5, and a linker sequence as set forth in SEQ ID NO:8, and a second polypeptide comprising an amino acid sequence the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:5, and a linker sequence as set forth in SEQ ID NO:15, and a second polypeptide comprising an amino acid sequence the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:5, and a linker sequence as set forth in SEQ ID NO:16, and a second polypeptide comprising an amino acid sequence the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:5, and a linker sequence as set forth in SEQ ID NO:23, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:4, the amino acid sequence as set forth in SEQ ID NO:5, and a linker sequence as set forth in SEQ ID NO:24, and a second polypeptide comprising an amino acid sequence which is the same as the first polypeptide.

In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:5, and a linker sequence as set forth in SEQ ID NO:8, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:5, and a linker sequence as set forth in SEQ ID NO:15, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:5, and a linker sequence as set forth in SEQ ID NO:16, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:5, and a linker sequence as set forth in SEQ ID NO:23, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:18, the amino acid sequence as set forth in SEQ ID NO:5, and a linker sequence as set forth in SEQ ID NO:24, and a second polypeptide comprising an amino acid sequence which is the same as the first polypeptide.

In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:5, and a linker sequence as set forth in SEQ ID NO:8, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:5, and a linker sequence as set forth in SEQ ID NO:15, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:5, and a linker sequence as set forth in SEQ ID NO:16, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:5, and a linker sequence as set forth in SEQ ID NO:23, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:19, the amino acid sequence as set forth in SEQ ID NO:5, and a linker sequence as set forth in SEQ ID NO:24, and a second polypeptide comprising an amino acid sequence which is the same as the first polypeptide.

In a specific embodiment, provided is a method of treating cancer in a subject. comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:11, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO12, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:20, and a second polypeptide comprising the amino acid sequence as set forth in SEQ ID NO:10. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:21, and a second polypeptide comprising the amino acid sequence as set forth in SEQ ID NO:9. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:17, and a second polypeptide comprising an amino acid sequence which is the same as the first polypeptide. In a specific embodiment, provided is a method of treating cancer in a subject, comprising administering to the subject a therapeutically effective amount of an IL-2 mutein or pharmaceutical composition thereof, wherein the IL-2 mutein comprises a first polypeptide comprising the amino acid sequence as set forth in SEQ ID NO:22, and a second polypeptide comprising an amino acid sequence which is the same as the first polypeptide.

In still another aspect, provided is a method of selectively activating CD8+ T cells or NK cells without significantly activating T regulatory cells in a subject, comprising administering to the subject in need thereof a therapeutically effective amount of any one of the various IL-2 muteins described herein or any one of the various pharmaceutical compositions described herein. In some embodiments, selectively activating CD8+ T cells or NK cells without significantly activating T regulatory cells can be measured by in vitro or ex vivo methods. In a specific embodiment, selectively activating CD8+ T cells or NK cells without significantly activating T regulatory cells can be measured by any methods known by a person of ordinary skills in the art, including but not limited to pSTAT5 assay or proliferation assay described in the examples provided herein.

In yet still another aspect, provided is a method of selectively activating cells that express IL-2 receptor βγ subunits but not significantly activating cells that express IL-2 receptor αβγ subunits compared to wild type IL-2 in a subject, comprising administering to the subject in need thereof a therapeutically effective amount of any one of the various IL-2 muteins described herein or any one of the various pharmaceutical compositions described herein. In some embodiments, selectively activating cells that express IL-2 receptor βγ subunits but not significantly activating cells that express IL-2 receptor αβγ subunits compared to wild type IL-2 can be measured by in vitro or ex vivo methods. In a specific embodiment, selectively activating cells that express IL-2 receptor βγ subunits but not significantly activating cells that express IL-2 receptor αβγ subunits compared to wild type IL-2 can be measured by any methods known by a person of ordinary skills in the art, including but not limited to pSTAT5 assay or proliferation assay described in the examples provided herein.

In yet still another aspect, provided is use of any one of the various IL-2 muteins described herein or any one of the various pharmaceutical compositions described herein to treat an IL-2-mediated disease in a subject.

In another aspect, provided is use of any one of the various IL-2 muteins described herein or any one of the various pharmaceutical compositions described herein for the preparation of a medicament to treat an IL-2-mediated disease in a subject.

In various uses of the IL-2 muteins described herein, the IL-2-mediated disease can be any cancer or infection described in this disclosure.

In certain various embodiments of methods or uses described herein, the IL-2 mutein or pharmaceutical composition comprising the IL-2 mutein is administered or used in combination with a second agent.

In certain embodiments, the IL-2 mutein and the second agent are administered or used simultaneously. In some embodiments, the IL-2 mutein and the second agent are administered or used sequentially. In certain embodiments, the IL-2 mutein and the second agent are administered or used through the same routes. In some embodiments, the IL-2 mutein and the second agent are administered or used through different routes.

In some embodiments, the second agent is a PD-1 antagonist, a PD-L1 antagonist, a CTLA4 antagonist, a LAG3 antagonist, a TIGIT antagonist, an ILT3 antagonist, an ILT4 antagonist, a PARP antagonist, a VEGF receptor antagonist, a FGF receptor antagonist, a HIF-2α antagonist, a BTK antagonist, an AKT antagonist, an ERK antagonist, a MEK antagonist, a CD27 agonist, a STING agonist, a chemotherapeutic agent, or an antibody-drug conjugate.

In one embodiment, the second agent is a PD-1 antagonist. In some embodiments, the PD-1 antagonist is selected from the group consisting of pembrolizumab, nivolumab, cemiplimab, pidilizumab (U.S. Pat. No. 7,332,582), dostarlimab, AMP-514 (MedImmune LLC, Gaithersburg, MD), PDR001 (U.S. Pat. No. 9,683,048), BGB-A317 (U.S. Pat. No. 8,735,553), and MGA012 (MacroGenics, Rockville, MD). In some embodiments, the PD-1 antagonist is any antibody, antigen binding fragment thereof, or variant thereof disclosed in U.S. Pat. Nos. 7,488,802, 7,521,051, 8,008,449, 8,354,509, 8,168,757, WO2004/004771, WO2004/072286, WO2004/056875, US2011/0271358, and WO 2008/156712, the disclosures of which are incorporated by reference herein in their entireties. In some embodiments, the PD-1 antagonist is pembrolizumab. In certain embodiments, the PD-1 antagonist is nivolumab. In other embodiments, the PD-1 antagonist is cemiplimab. In yet other embodiments, the PD-1 antagonist is pidilizumab. In yet other embodiments, the PD-1 antagonist is dostarlimab.

Other PD-1 antagonists useful in various methods, kits, and uses described herein include an immunoadhesion molecule that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, the disclosures of which are incorporated by reference herein in their entireties. Specific fusion proteins useful as the PD-1 antagonist in various methods, kits, and uses described herein include AMP-224 (also known as B7-DCIg), which is a PD-L2-Fc fusion protein and binds to human PD-1.

In another embodiment, the second agent is a PD-L1 antagonist. Examples of PD-L1 antagonists that can be used in various methods, kits, and uses described herein are disclosed in WO2013/019906, WO2010/077634, and U.S. Pat. No. 8,383,796, the disclosures of which are incorporated by reference herein in their entireties. Specific PD-L1 antagonist includes atezolizumab, durvalumab, avelumab, BMS-936559, and an antibody comprising the heavy chain and light chain variable regions of SEQ ID NO:20 and SEQ ID NO:21, respectively, of WO2013/019906. In some embodiments, the PD-L1 antagonist is atezolizumab. In certain embodiments, the PD-L1 antagonist is durvalumab. In other embodiments, the PD-L1 antagonist is avelumab.

In various embodiments, the PD-1 or PD-L1 antagonist comprises a variant of the amino acid sequences of the anti-human PD-1 antibody (e.g., pembrolizumab, nivolumab, dostarlimab, or cemiplimab) or anti-human PD-L1 antibody (e.g., atezolizumab, durvalumab, or avelumab) described herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the PD-1 or PD-L1 antagonist comprises a V_{L} domain with at least 97%, 95%, 90%, 85%, 80%, 75% or 50% sequence homology to one of the V_{L} domains of the anti-human PD-1 or anti-human PD-L1 antibodies described herein, and exhibits specific binding to PD-1 or PD-L1. In another embodiment, the PD-1 or PD-L1 antagonist comprises a V_{H} domain with at least 97%, 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1. In yet another embodiment, the PD-1 or PD-L1 antagonist comprises a V_{L} domain with at least 97%, 95%, 90%, 85%, 80%, 75% or 50% sequence homology to one of the V_{L} domains of the anti-human PD-1 or anti-human PD-L1 antibodies described herein and a V_{H} domain with at least 97%, 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1.

In one embodiment, the second agent is a CTLA4 antagonist. In another embodiment, the second agent is a LAG3 antagonist. In yet another embodiment, the second agent is a TIGIT antagonist. In still another embodiment, the second agent is an ILT3 antagonist. In yet still another embodiment, the second agent is an ILT4 antagonist. In one embodiment, the second agent is a PARP antagonist. In another embodiment, the second agent is a VEGF receptor antagonist. In yet another embodiment, the second agent is a FGF receptor antagonist. In still another embodiment, the second agent is a HIF-2a antagonist. In yet still another embodiment, the second agent is a BTK antagonist. In one embodiment, the second agent is an AKT antagonist. In another embodiment, the second agent is an ERK antagonist. In yet another embodiment, the second agent is a MEK antagonist. In still another embodiment, the second agent is a CD27 agonist. In yet still another embodiment, the second agent is a STING agonist. In one embodiment, the second agent is a chemotherapeutic agent. In another embodiment, the second agent is an antibody-drug conjugate.

### Nucleic Acids, Expression Vectors, Cells, and Methods of Making IL-2 Muteins

Also provided herein are isolated nucleic acids and vectors comprising polynucleotide sequences encoding such IL-2 muteins disclosed herein, cells (e.g., host cells) comprising such isolated nucleic acids or vectors, and methods of making such IL-2 muteins.

In one aspect, provided is an isolated nucleic acid, encoding a polypeptide of an IL-2 mutein described herein. In one embodiment, the isolated nucleic acid encodes any one of the various first polypeptides of the various IL-2 muteins described herein. In another embodiment, the isolated nucleic acid encodes any one of the various second polypeptides of the various IL-2 muteins described herein. In yet another embodiment, the isolated nucleic acid encodes any one of the various first polypeptides and the various second polypeptides of the various IL-2 muteins described herein. In still another embodiment, the isolated nucleic acid encodes one or more polypeptides disclosed in Table 9.

In another aspect, provided is a first isolated nucleic acid that encodes the first polypeptide and a second isolated nucleic acid that encodes the second polypeptide of an IL-2 mutein described herein.

In another embodiment, the isolated nucleic acids further encode a signal sequence.

In another aspect, provided is an expression vector comprising one or more of the various isolated nucleic acids disclosed herein, wherein the nucleic acid(s) is operably linked to control sequences that are recognized by a host cell when the host cell is transfected with the expression vector.

In yet another aspect, provided is host cell comprising one or more of the various isolated nucleic acids or the various expression vectors disclosed herein. In some embodiments, the host cell comprises one or more of the various isolated nucleic acids disclosed herein. In other embodiments, the host cell comprises one or more of the various expression vectors disclosed herein. In yet other embodiments, the host cell comprises one of the various expression vectors disclosed herein, wherein the expression vector comprises one or more of the various isolated nucleic acids disclosed herein. In still other embodiments, the host cell comprises a first expression vector comprising a first nucleic acid that encodes the first polypeptide and a second expression vector comprising a second nucleic acid that encodes the second polypeptide of the IL-2 mutein described herein.

In still another aspect, provided is a method of producing any one of the various IL-2 muteins described herein. In one embodiment, the method comprises culturing any of the various host cells described herein under conditions wherein the IL-2 mutein is expressed. In another embodiment, the method comprises expressing any one of the various expression vectors described herein under conditions wherein the IL-2 mutein is expressed. In yet another embodiment, the method comprises expressing any one of the various isolated nucleic acids described herein under conditions wherein the IL-2 mutein is expressed.

In certain embodiments of the various methods of making the IL-2 muteins, the method further comprises isolating the IL-2 muteins from the host cell or culture medium, or in vitro expression system.

Mammalian cell lines available as hosts for expression of the IL-2 muteins disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Other cell lines that may be used are insect cell lines, such as Sf9) cells, amphibian cells, bacterial cells, plant cells and fungal cells. Various modifications can be introduced into the genome of these cell lines (e.g., glutamine synthetase knockout, auxotrophic mutations, etc.) to achieve desired properties of the host cells and/or desired properties of the expressed IL-2 muteins.

When recombinant expression vectors encoding the IL-2 mutein are introduced into host cells, the IL-2 mutein is produced by culturing the host cells for a period of time sufficient to allow for expression of the IL-2 mutein in the host cells or, more preferably, secretion of the IL-2 mutein into the culture medium in which the host cells are grown.

IL-2 muteins can be recovered from the culture medium using standard protein purification methods.

In general, glycoproteins produced in a particular cell line or transgenic animal will have a glycosylation pattern that is characteristic for glycoproteins produced in the cell line or transgenic animal. Therefore, the particular glycosylation pattern of an IL-2 mutein will depend on the particular cell line or transgenic animal used to produce the IL-2 mutein. The disclosures of various IL-2 muteins or isolated nucleic acids encoding such IL-2 muteins are independent of the glycosylation pattern the IL-2 muteins may have.

### Pharmaceutical Compositions and Administration

In another aspect, provided is a composition comprising any one of the IL-2 muteins described herein and a pharmaceutically acceptable carrier.

In some embodiments, the composition further comprises an additional agent.

In particular embodiments, the additional agent is an agent effective to treat the same disorder as the IL-2 muteins disclosed herein are being used to treat. In some embodiments, the additional agent is an agent effective to relieve side effects of the IL-2 muteins disclosed herein.

To prepare pharmaceutical or sterile compositions of the IL-2 muteins described herein, the IL-2 mutein is admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

Toxicity and therapeutic efficacy of the IL-2 mutein compositions, administered alone or in combination with another agent, can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In a further embodiment, a composition comprising an IL-2 mutein disclosed herein is administered to a subject in accordance with the Physicians' Desk Reference 2003 (Thomson Healthcare; 57th edition (Nov. 1, 2002)).

The mode of administration of the IL-2 muteins and compositions of the invention can vary. Suitable routes of administration include oral, rectal, transmucosal, intestinal, parenteral, intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the IL-2 mutein can be administered by an invasive route such as by injection. In further embodiments, the IL-2 mutein, or pharmaceutical composition thereof, is administered intravenously, subcutaneously, intrathecally, intramuscularly, or intracerebrally. In one specific embodiment, the IL-2 mutein, or pharmaceutical composition thereof, is administered intravenously. In another specific embodiment, the IL-2 mutein, or pharmaceutical composition thereof, is administered subcutaneously.

Compositions can be administered with medical devices known in the art. For example, a pharmaceutical composition of the invention can be administered by injection with a hypodermic needle, including, e.g., a prefilled syringe or autoinjector.

The pharmaceutical compositions disclosed herein may also be administered by infusion.

The administration regimen depends on several factors, including the serum or tissue turnover rate of the IL-2 mutein, the level of symptoms, the immunogenicity of the IL-2 mutein, and the accessibility of the target cells in the biological matrix. Preferably, the administration regimen delivers sufficient IL-2 mutein to effect improvement in the target disease state, while simultaneously minimizing undesired side effects. Accordingly, the amount of biologic delivered depends in part on the particular IL-2 mutein and the severity of the condition being treated. Guidance in selecting appropriate doses is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects.

As previously described, the IL-2 mutein may be coadministered with one or more additional agents. The IL-2 mutein can be linked to the agent (such as a conjugate) or can be administered separately from the agent. In the latter case (separate administration), the IL-2 mutein can be administered before, after, or concurrently with the agent or can be co-administered with other known therapies.

In some embodiments, the IL-2 mutein and one or more additional agents (e.g., the second agent described in this disclosure) are in the same pharmaceutical composition. In other embodiments, the IL-2 mutein and one or more additional agents (e.g., the second agent described in this disclosure) are in different pharmaceutical compositions.

### Kits

Also provided herein are kits comprising an IL-2 mutein provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. In some embodiments, the kits include a second agent as disclosed herein. Each component of the kit can be enclosed within an individual container, and all of the various containers can be within a single package. Kits can also be designed for cold storage.

### General Methods

Standard methods in molecular biology are described in Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLE 1. DESIGNING, CLONING, EXPRESSION, AND PURIFICATION OF IL-2 MUTEINS

The strategy for generating the desired IL-2Rβγ-biased and half-life extension (HLE) profile for IL-2 focused on two modifications to the IL-2 protein. First, to obtain the desired bias, site-specific mutations were made on IL-2 to disrupt IL-2Rα (CD25) binding. Second, to extend half-life, IL-2 muteins were fused to an effector function-null human IgG1 Fc to increase the molecular size and mediate binding to FcRn to enable intracellular recycling.

Multiple formats of Fc-IL-2 fusions were tested for expression, stability, and activity. These included bivalent IL-2 fusions and monovalent IL-2 fusions using Knobs-into-Holes (KIH) technology for forming Fc heterodimers. Additionally, both N- and C-terminal fusions were tested using various linker designs, including multiple GGGGS linkers with different lengths. Flexible polypeptide linkers composed of glycine and serine are important components of engineered multidomain proteins and were used in the design of the novel molecules to provide separation between the Fc domain and the mutated IL-2.

The following Table 1 shows an exemplary study on the high molecular weight (HMW) protein present in single step protein A chromatography purified IL2-Fc fusion proteins with different linkers. “C10” indicates IL2 is fused to Fc on the C-terminus with a 10 amino acid GGGGSGGGGS linker. “N10” indicates IL2 is fused to Fc on the N-terminus with a 10 amino acid GGGGSGGGGS linker. “C3” indicates IL2 is fused to Fc on the C-terminus with a 3 amino acid GGS linker. “N3” indicates IL2 is fused to Fc on the N-terminus with a 3 amino acid GGS linker. This screening was done using wild type (WT) IL2 containing the T3A mutation. Transient expression levels in both CHO and HEK were similar across the constructs (between 50 mg/L-100 mg/L).

TABLE 1
HMW percentage after single step protein A chromatography purification

| | % HMW (bivalent) | % HMW (monovalent) |
|---|---|---|
| C10 | 2 | 2 |
| C3 | 2 | 5 |
| N10 | 12 | 7 |
| N3 | 28 | 7 |

Seven additional Y45R monovalent variants were generated and assessed, C-terminal IL-2: linkers of length 5, 8, and 12 (SEQ ID NO: 25, SEQ ID NO: 25, and SEQ ID NO: 27 respectively), N-terminal IL-2: linkers of length 5 and 10 (SEQ ID NO: 28, and SEQ ID NO: 29 respectively), and C-terminal IL-2 reverse KIH (hole instead of knob): linkers of length 5 and 10 (SEQ ID NO: 30, and SEQ ID NO: 31 respectively). The additional variants all had similarly low % HMW (varied from not detectable to 1%) shown in Table 2. The following Table 2 shows an exemplary study on the high molecular weight (HMW) protein present in single step protein A chromatography purified IL-2 Y45R variants -Fc fusion proteins with different linkers. “C5” indicates IL-2 Y45R variant is fused to Fc on the C-terminus with a 5 amino acid GGGGS linker. “C8” indicates IL-2 Y45R variant is fused to Fc on the C-terminus with a 8 amino acid GGGSGGGS linker. “C12” indicates IL-2 Y45R variant is fused to Fc on the C-terminus with a 12 amino acid GSGGGGSGGGGS linker. “N5” indicates IL-2 Y45R variant is fused to Fc on the N-terminus with a 5 amino acid GGGGS linker. “N10” indicates IL-2 Y45R variant is fused to Fc on the N-terminus with a 10 amino acid GGGGSGGGGS linker. “C5 HIK” indicates IL-2 Y45R variant is fused to Fc on the C-terminus having a reverse knob in hole configuration with a 5 amino acid GGGGS linker. “C10 HIK” indicates IL-2 Y45R variant is fused to Fc on the C-terminus having a reverse knob in hole configuration with a 10 amino acid GGGGGGGGS linker. This screening was done using IL-2 Y45R variants.

TABLE 2
HMW percentage after single step protein A chromatography purification

| | % HMW (monovalent) |
|---|---|
| C5 | 1 |
| C8 | 0.6 |
| C10 | .04 |
| N5 | Not detected |
| N10 | Not detected |
| C5 | Not detected |
| HIK | |
| C10 | Not detected |
| HIK | |

In addition, monovalent and bivalent IL-2 mutein Fc fusion molecules were tested in various functional assays, including binding assay (as in Example 2) and in vitro activity assays (as in Example 3). In an exemplary receptor engagement study (as in Example 3), compared to bivalent molecules, monovalent molecules (e.g., Y45R-IL-2 Fc, R38Q/Y45F-IL-2 Fc) exhibited more significant differentiation between engaging the IL-2Rαβ and engaging IL-2Rβγ subunits (Table 3).

TABLE 3
EC50 values of bivalent and monovalent IL-2 mutein Fc fusion molecules
engaging IL-2Rαβ or IL-2Rβγ subunits

| | IL-2Rαβ | IL-2Rβγ |
|---|---|---|
| | Ave. EC50 (nM) | Ave. EC50 (nM) |
| | N = 3 | N = 3 |
| Y45R-IL-2 Fc, bivalent | 30 | 2.3 |
| Y45R-IL-2 Fc, monovalent | 501 | 1.2 |
| R38Q/Y45F-IL-2 Fc, bivalent | 7 | 3.5 |
| R38Q/Y45F-IL-2 Fc, monovalent | 400 | 2.0 |

The decision to use a monovalent C-terminal fusion of IL2 was made for a few reasons. Bivalent N-terminal IL-2 constructs were eliminated based on increased levels of aggregation indicated by >10% HMW in analytical SEC. Monovalent IL2 was chosen for functional reasons with the goal of maintaining similar binding kinetics to WT monovalent IL2 and ease of functional assessment in binding and in vitro activity assays. The 10 GS linker was chosen to leave additional space between the Fc and the IL-2 mutein. Additionally, functional activity relative to WT non-Fc fused IL2 was considered.

The final molecule was selected based on biophysical properties and the preservation of IL-2 activity mediated through binding to IL-2Rβγ-containing receptors. It contains human IgG1 with LALADS mutations fused to a monovalent C-terminal IL-2 using a GGGGSGGGGS (SEQ ID NO:8) linker. A T3A mutation in IL-2 removed a site likely to be glycosylated when the molecules would be expressed in mammalian cells. Cysteine 125 has been mutated to serine, a mutation applied in aldesleukin to reduce disulfide mispairing and aggregation. The HLE molecule described above was used as a platform to introduce amino acid point mutations including but not limited to sites on the IL-2Rα interacting surface as defined by reported co-crystal structures of IL-2 bound to IL-2Rα (CD25), IL-2Rβ (CD122), and IL-2Rγ (CD132). Site-specific mutations on IL-2 were identified through computational structure-based design. Missing loops on IL-2 in the structure were homology modeled, and then IL-2 residues proximal to IL-2Rα were identified for computational single and double saturation mutagenesis. The results informed a first-round selection of two dozen single point mutants, two double mutants, and mutants to disrupt secondary structure elements at the interface. Variants were generated as Fc fusions using Expi293 cells, and then screened for binding to alpha and beta receptors and cell-based assays. In a second round of mutagenesis, successful variants from the first round were combined as double, triple, and quadruple mutants. Following further screening and characterization, the Y45R variant, where amino acid numbering refers to the position in the IL-2 molecule, was selected as the lead variant and scaled up in ExpiCHO-S cells.

Polynucleotides encoding various IL-2 muteins were synthesized and cloned into the PTT5 plasmid (Canadian National Research Council). ExpiCHO or Expi293 cells growing in Gibco ExpiCHO or Expi293 Expression Medium in suspension were transiently transfected with the PTT5 constructs comprising the polynucleotides encoding the various IL-2 muteins, using commercially available reagent ExpiFectamine and protocols (Thermo-Fisher). In brief, cells were transfected day 0 using 1 μg total DNA per 1 mL cells with viability>95% measured using a Vi-Cell (Beckman-Coulter). For expressing bivalent IL-2 muteins, a PTT5 construct encoding an IL-2 mutein containing a Fc variant Fc-1 (SEQ ID NO:5) was transfected. For some embodiments of expressing monovalent IL-2 muteins, a first PTT5 construct encoding an IL-2 mutein containing a Fc variant Fc-2 (SEQ ID NO:6) and a second PTT5 construct encoding a Fc variant Fc-5 (SEQ ID NO:10) were co-transfected, and the ratio of the first PTT5 construct to the second PTT5 construct was 3:1. In other embodiments of expressing monovalent IL-2 muteins, a first PTT5 construct encoding an IL-2 mutein containing a Fc variant Fc-3 (SEQ ID NO:7) and a second PTT5 construct encoding a Fc variant Fc-4 (SEQ ID NO:9) were co-transfected, and the ratio of the first PTT5 construct to the second PTT5 construct is 3:1. Cultures were harvested on day 5 for Expi293 cells and between day 8 and day 12 depending on a cell viability greater than 80% for ExpiCHO cells. Monovalent or bivalent IL-2 muteins were purified from clarified supernatant using Protein A chromatography (mAbSelect Sure LX, GE Healthcare) on AKTA FPLC systems (GE Healthcare). Following loading, resin was washed with 20 column volumes of PBS and the IL-2 muteins were eluted using 20 mM sodium acetate, pH 3.5. For bivalent IL-2 muteins, anion exchange (Capto Q, GE Healthcare) pass through following Protein A chromatography was typically sufficient for high homogeneity protein. For monovalent IL-2 muteins, following initial Protein A purification and anion exchange pass-through, cation exchange chromatography (Capto S) was used to separate the desired monovalent heterodimer IL-2 mutein from the contaminants, such as Fc variant homodimer, IL-2 mutein monomer and/or IL-2 mutein homodimer, to achieve greater than 95% monovalent heterodimer. Different variants required different salt and pH conditions for separation. In certain cases, size exclusion chromatography was further used to achieve >95% monovalent heterodimer.

### EXAMPLE 2. DETERMINATION OF BINDING AFFINITY OF IL-2 MUTEINS TO HUMAN, CYNOMOLGUS, RHESUS, MOUSE, AND RAT IL2-Rα OR IL2-Rβ BY SURFACE PLASMON RESONANCE

A surface plasmon resonance (SPR) assay on a Biacore T200 (Cytiva) instrument was used to determine the binding affinities of IL-2 muteins against polyhistidine-tagged IL-2 receptors. Biacore T200 Evaluation Software was used to fit each titration series to a 1:1 binding model or steady state affinity. The association rate constant (kₒₙ, M⁻¹ s⁻¹) and dissociation rate constant (k_{off}, s⁻¹) were determined for each set of titrations and used to calculate the dissociation constant, K_{D}=k_{off}/kₒₙ, of each IL-2 mutein against each receptor.

To measure the affinity of IL-2 muteins for IL-2 receptors, two orientations were utilized, firstly the receptors were captured on an anti-histidine surface followed by the IL-2 Fc fusion as analyte, or secondly the IL-2 Fc fusion was captured on an anti-human IgG Fc antibody surface followed by the IL-2 receptor as analyte.

As shown in Table 4, the affinity of WT-IL-2 Fc for all species of IL-2Rα is between 28 nM and 87 nM. Y45R-IL-2 Fc does not show detectable binding to human, cynomolgus, rhesus, and mouse IL-2Rα when assayed at 3 μM. Y45R-IL-2 Fc does show 31-fold decreased affinity to rat IL-2Rα when compared to WT-IL-2 Fc. The affinities of WT-IL-2 Fc and Y45R-IL-2 Fc for human and cynomolgus/rhesus IL-2Rβ are similar and within 1.5-fold of each other. They both show weak binding to rat IL-2Rβ. “WT-IL-2 Fc” and “Y45R-IL-2 Fc” tested here both contain a T to A substitution corresponding to position 3 and a C to S substitution corresponding to position 125 of wild type human IL-2 (SEQ ID NO:13). “Y45R-IL-2 Fc” further contains a Y to R substitution corresponding to position 45 of wild type human IL-2 (SEQ ID NO:13).

TABLE 4
Binding affinities of WT-IL-2 Fc and Y45R-IL-2 Fc
to multiple species IL-2Rα or IL-2Rβ by SPR

| | | | | | Y45R-IL-2 Fc | |
|---|---|---|---|---|---|---|
| IL-2R | | | WT-IL-2 Fc | | | KD/KD |
| Subunit | Species | Tag | KD (M)¹ | KD/KD (hu) | KD (M)¹ | (hu) |
| alpha | Human | His | 2.8E−08 | 1.0 | NB² | n/a³ |
| | Cynomolgus | | 3.0E−08 | 1.1 | NB | n/a |
| | Rhesus | | 4.9E−08 | 1.8 | NB | n/a |
| | Mouse | | 2.9E−08 | 1.0 | NB | n/a |
| | Rat | | 8.7E−08 | 3.1 | 2.7E−06 | n/a |
| beta | Human | His | 9.4E−07 | 1.0 | 1.3E−06 | 1.0 |
| | Cynomolgus/ | | 1.3E−06 | 1.4 | 2.0E−06 | 1.5 |
| | Rhesus⁴ | | | | | |
| | Mouse | | ND⁵ | n/a | ND | n/a |
| | Rat | | WB⁶ | n/a | WB | n/a |

1. Number of runs for all conditions = 2;
2. NB (no binding observed at 3 μM);
3. n/a (not applicable);
4. There is 100% amino acid identity between cyno and rhesus IL-2Rβ extracellular region.
5. ND (not done), mouse IL-2Rβ reagent showed no detectable binding to positive control, mouse IL-2).
6. WB (weak binding), signal is too low to generate a KD, but a small amount of binding is observed.

### EXAMPLE 3. Y45R-IL-2 FC VS. WT-IL-2 FC & ALDESLEUKIN IN RECEPTOR ENGAGEMENT AND PSTAT5 ACTIVATION ASSAYS

Aldesleukin, WT-IL-2 Fc and Y45R-IL-2 Fc were assessed for bias to CD122 (IL-2Rβ) in the U2OS (human bone osteosarcoma epithelial) cell line that has been engineered to capture the engagement of the IL-2Rαβ or βγ subunits. This assay employs Enzyme Fragment Complementation Technology (EFC) technology (Eurofins DiscoverX Products, LLC, Fremont, CA) in which IL-2R subunits are tagged with inactive enzyme fragments. When IL-2 binds and triggers the dimerization of the IL-2R subunits, the enzyme fragments are combined and subsequently activated to hydrolyzes a substrate that produces a chemiluminescence signal.

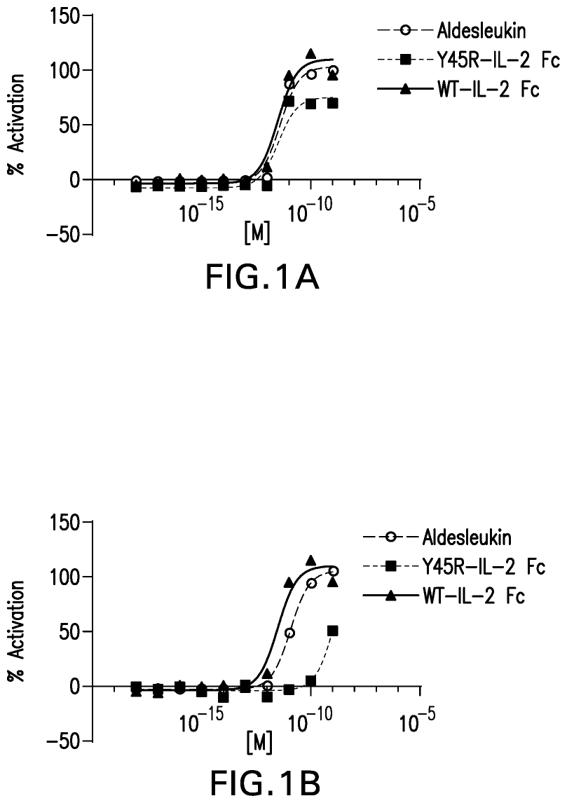

In U2OS [βγ] cells, aldesleukin, WT-IL-2 Fc and Y45R-IL-2 Fc demonstrated a dose-dependent increase in βγ dimerization with Y45R-IL-2 Fc possessing a reduced Emax (FIG. 1A). In contrast, in U2OS [αβ] cells aldesleukin and WT-IL-2 Fc exhibited a dose-dependent increase in dimerization, and Y45R-IL-2 Fc demonstrated a right-shifted or no response confirming the bias in IL-2 receptor binding (FIG. 1B and Table 5). These data combined with Biacore results confirm the CD122 bias of Y45R-IL-2 Fc.

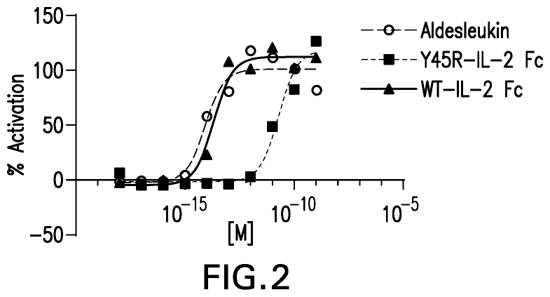

Y45R-IL-2 Fc, along with aldesleukin and WT-IL-2 Fc, was also assessed in CTLL-2 cells (mouse cytotoxic T-cell line) to determine its functional bias in context to activation of IL-2 receptor proximal downstream signaling through pSTAT5. CTLL-2 expresses IL-2Rα, β and γ subunits. Aldesleukin and WT-IL-2 Fc demonstrated a dose-dependent activation of pSTAT5 at 30 min (FIG. 2). Y45R-IL-2 Fc exhibited a reduced potency with a right shifted response to activate phospho-STAT5 at the concentrations tested (FIG. 2 and Table 4). Collectively, these results with the U2OS (αβ) and U2OS (βγ) dimerization assays demonstrate that Y45R-IL-2 Fc is a biased ligand and functional in activation of IL-2 receptor through IL-2Rβγ subunits.

TABLE 5
Aldesleukin, WT-IL-2 Fc and Y45R-IL-2 Fc in U2OS Receptor
Engagement and CTLL-2 pSTAT5 Activation Assays

| | U2OS-IL-2R | U2OS-IL-2R | CTLL-2 |
|---|---|---|---|
| | (αβ) | (βγ) | pSTAT5 |
| Compound | EC₅₀ ± S.D. (nM) | | |
| Aldesleukin | 4.06 ± 5.3 | 2.5 ± 0.98 | 0.08 ± 0.18 |
| Y45R-IL-2 Fc | >1000 | 3.58 ± 0.25 | 17.18 ± 2.2 |
| WT-IL-2 Fc | 3.8 ± 0.98 | 14.2 ± 10.05 | 0.03 ± 0.01 |

Seven additional Y45R monovalent variants were generated and assessed, C-terminal IL-2: linkers of length 5, 8, and 12 (SEQ ID NO: 25, SEQ ID NO: 26, and SEQ ID NO: 27 respectively), N-terminal IL-2: linkers of length 5 and 10 (SEQ ID NO: 28, and SEQ ID NO: 29 respectively), and C-terminal IL-2 reverse KIH (hole instead of knob): linkers of length 5 and 10 (SEQ ID NO: 30, and SEQ ID NO: 31 respectively). The additional variants all had similar Biacore affinity to IL-2Ra (weak binding) and IL-2Rb (0.98-2.12 μM). As for pSTAT5 activity, the C-terminal IL-2 variants had activity similar to WT (4.5-5.4 nM), the N-term IL-2 variants had weaker activity (30-84 nM) and the Reverse KIH constructs had weaker activity compared to C-term fusions (13-19 nM).

### EXAMPLE 5. Y45R-IL-2 FC SHOWED REDUCTION IN ACTIVITY ON TREGS AND CD8⁺ CYTOTOXIC T LYMPHOCYTES FROM HUMAN AND RHESUS MONKEY IN COMPARISON TO WT-IL-2 FC

The potency of Y45R-IL-2 Fc and the non-biased WT-IL-2 Fc in stimulating the proximal signaling of the IL-2R complex was assessed in Tregs (expressing IL-2Rαβγ), CD8 (expressing IL-2Rβγ) and NK cells (expressing IL-2Rβγ) by measuring pSTAT5 in fresh whole blood samples from multiple human and rhesus monkey donors using flow cytometry analysis; pSTAT5 EC50 values were generated for each cell type.

Fresh rhesus monkey and human whole blood samples (100 μl/well) were treated with Y45R-IL-2 Fc or WT-IL-2-Fc for 30 min (37° C. 5% CO₂) followed by surface staining for CD8+ T and NK cell lineage markers at room temperature for 20 mins. Afterwards, blood samples were lysed, fixed, and permeabilized to stain for phospho-STAT5, CD4, CD8 and FOXP3 at room temperature for 1 hr. Samples were washed and analyzed using the BD LSR-Fortessa. Non-linear regression analysis was performed using Prism software.

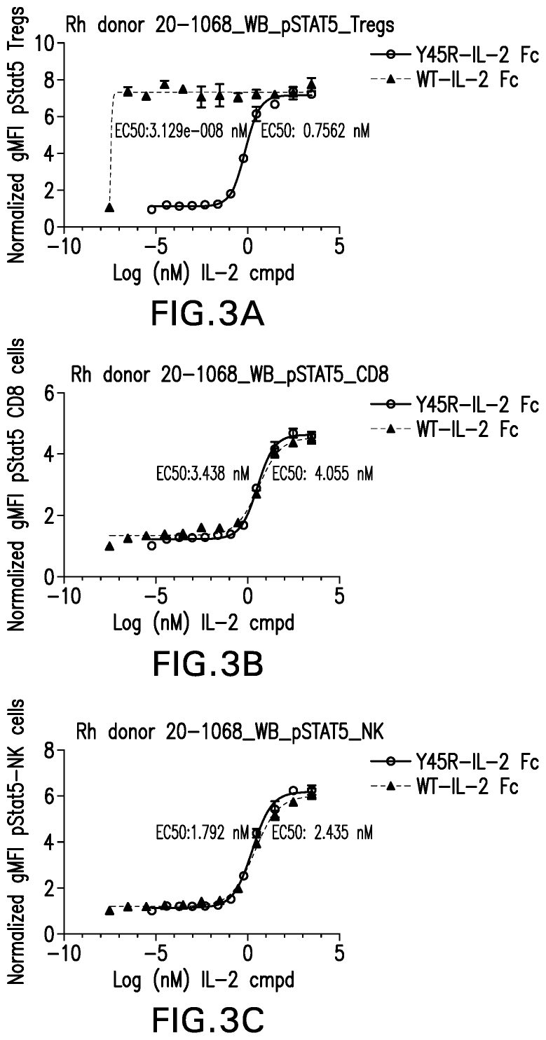

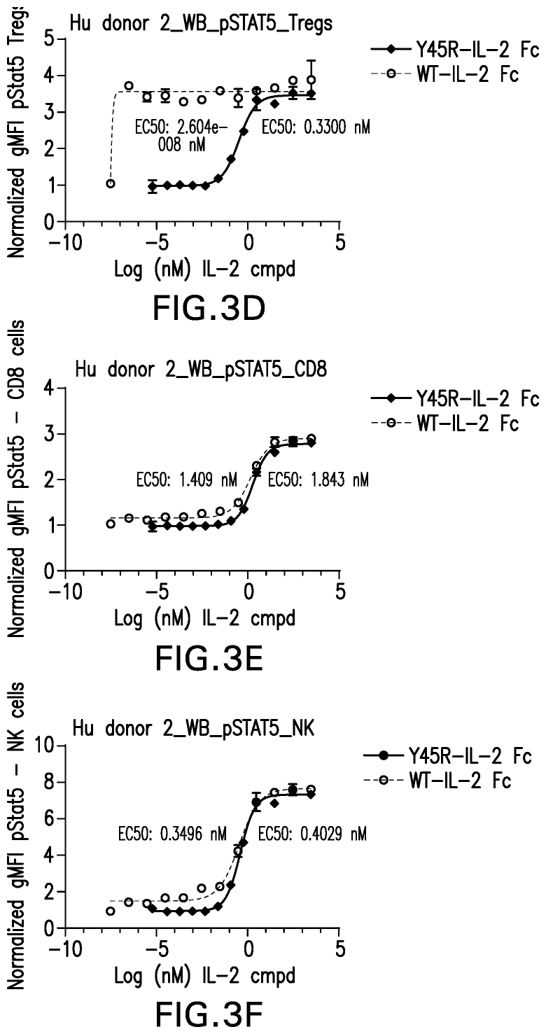

Compared to WT-IL-2 Fc, Y45R-IL-2 Fc showed a robust reduction (>12000 fold decrease) of Treg activation in both species, as measured by average EC50 values of normalized pSTAT5 gMFI, while the pSTAT5 activation in NK and CD8⁺ T cells remained comparable (changes less than 2 fold) between Y45R-IL-2 Fc and WT-IL-2-Fc (FIGS. 3A-3F). The same biased activity of Y45R-IL-2 Fc was observed in mouse splenocytes (data not shown).

### EXAMPLE 6. Y45R-IL-2 FC TREATMENT IN NON-TUMOR BEARING MICE RESULTED IN ROBUST AND SUSTAINED PHOSPHORYLATION OF STAT5 IN AND EXPANSION OF CD8+ T CELL AND NK CELL POPULATIONS

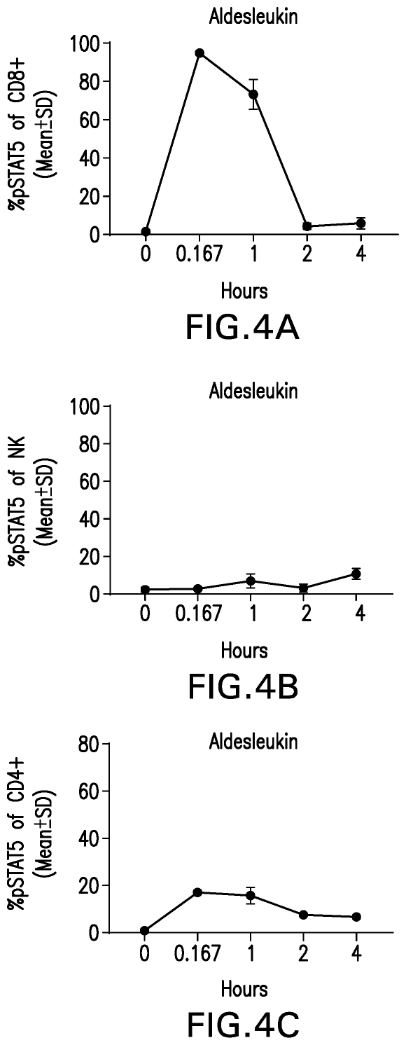

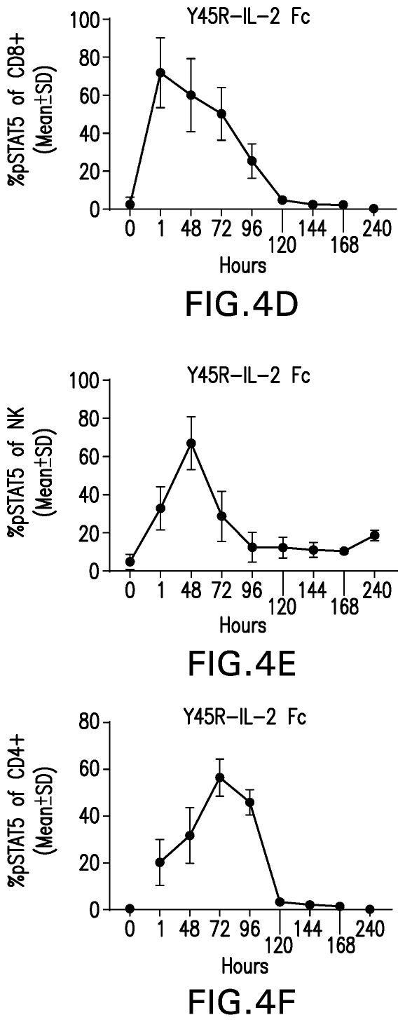

The phosphorylation of STAT5 in different immune cell subsets by aldesleukin and Y45R-IL-2 Fc was assessed in non-tumor bearing C57BL/6 mice (FIGS. 4A-4F). All mice were dosed once and phosphorylated STAT5 in the blood was assessed at multiple times points over the course of 10 days, except for aldesleukin due to its known short half-life. Aldesleukin induced pSTAT5 in nearly 100% of CD8⁺ T cells and about 20% of CD4⁺ T cells within 10 mins of administration and was back to baseline by 2 hours post-injection. Less than 20% of NK cells were pSTAT5⁺ at both 1- and 4-hours post-injection. Y45R-IL-2 Fc induced pSTAT5 in 70% of CD8+ T cells by 1-hour post-injection and sustained pSTAT5 through 120 hours. Additionally, pSTAT5 was induced in ˜70% of NK cells at their peak and sustained through 96 hours and pSTAT5 was induced in 55% of CD4⁺ T cells that peaked at 72 and subsided by 120 hours.

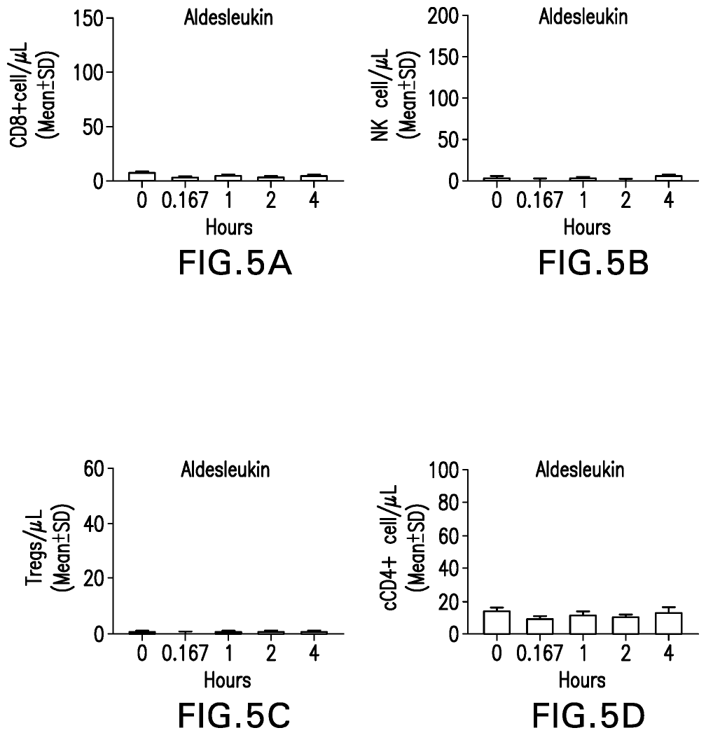

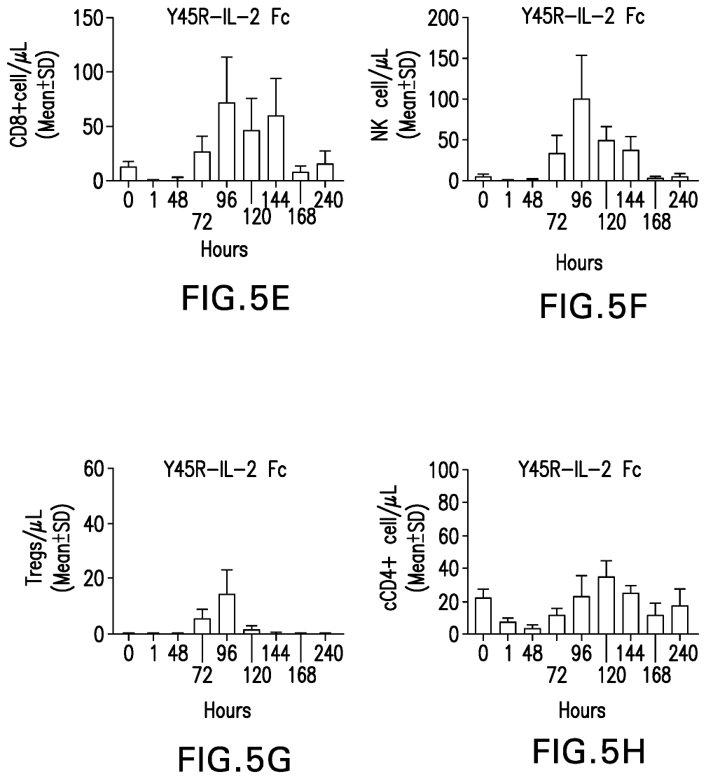

In parallel, T cell and NK cell populations were identified and quantified at each time point (FIGS. 5A-5H). Due to its short half-life, there was no increase in the blood cell concentrations in mice treated with aldesleukin over this time period. In mice treated with Y45R-IL-2 Fc, there was a robust expansion of CD8⁺ T cells and NK cells after a period of contraction that peaked at 96 hours, at 73 and 101 cells/μL respectively, and returned to baseline by 168 hours post-administration. Similarly, Tregs were increased in the blood to 15 cells/μL by 96 hours and returned to baseline by 144 hours post-injection while conventional CD4⁺ T cells stayed similar to baseline after an initial contraction.

### EXAMPLE 7. IN VIVO EFFECT OF Y45R-IL-2 FC ON PSTAT5 IN RHESUS CD8⁺ T, NK AND TREG CELL POPULATIONS RELATIVE TO WT-IL-2 FC

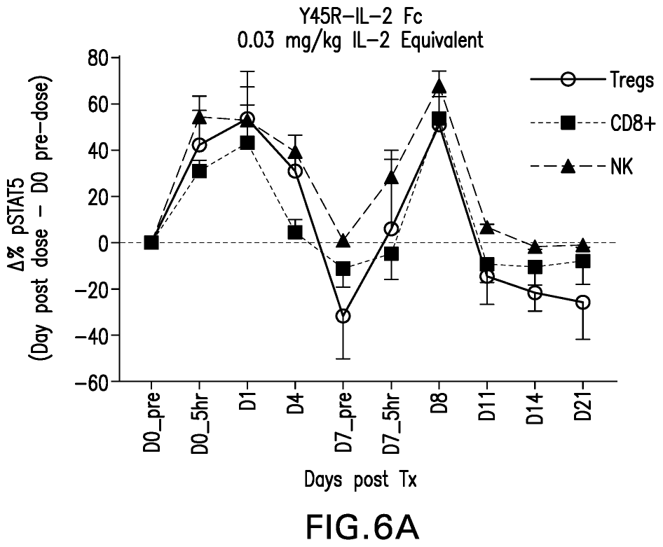

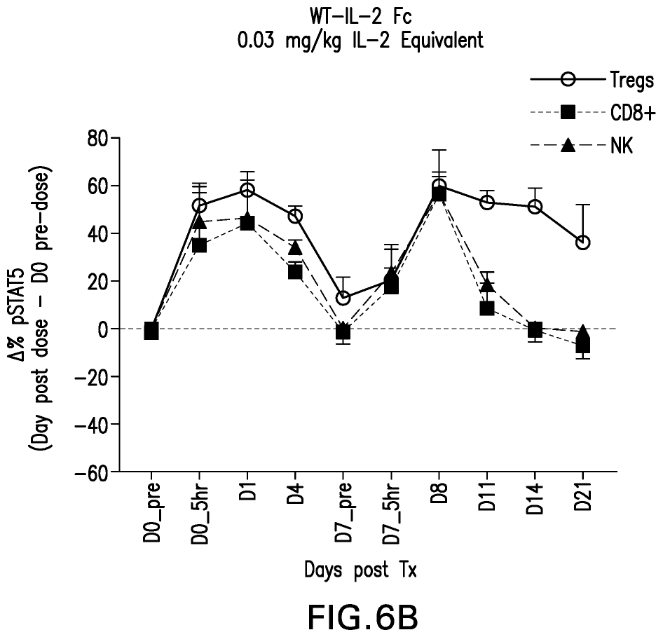

Induction of pSTAT5 in CD8⁺ T, NK and T regs by Y45R-IL-2 Fc and WT-IL-2 Fc was assessed in rhesus NHPs (FIGS. 6A and 6B). Rhesus NHPs (N=3/group) were given two doses, seven days apart, of Y45R-IL-2 Fc or WT-IL-2 Fc. Phospho-STAT5 expression levels in the blood were then determined at various times points over the course of 21 days. Y45R-IL-2 Fc induced a sustained increase (%) in pSTAT5 levels in CD8⁺ T and NK cells up to 4 days (96 hrs) and 7 days (168 hrs), respectively, after the 1^{st} dose and up to 4 days (96 hrs) in both subsets after the 2^{nd} dose. These results were comparable to pSTAT5 activation in CD8⁺ T and NK cells by WT-IL-2 Fc. Y45R-IL-2 Fc's capacity for bias towards the IL-2Rβγ binding was seen after the 2^{nd} dose, which resulted in comparable pSTAT5 induction in Tregs, CD8⁺ T and NK cells. On the other hand, WT-IL-2 Fc induced pSTAT5 at a significantly greater level in Tregs than CD8⁺ T and NK cells after the 2nd dose.

### EXAMPLE 8. PHARMACODYNAMIC EFFECT OF Y45R-IL-2 FC ON B16F10 TUMOR-BEARING MICE

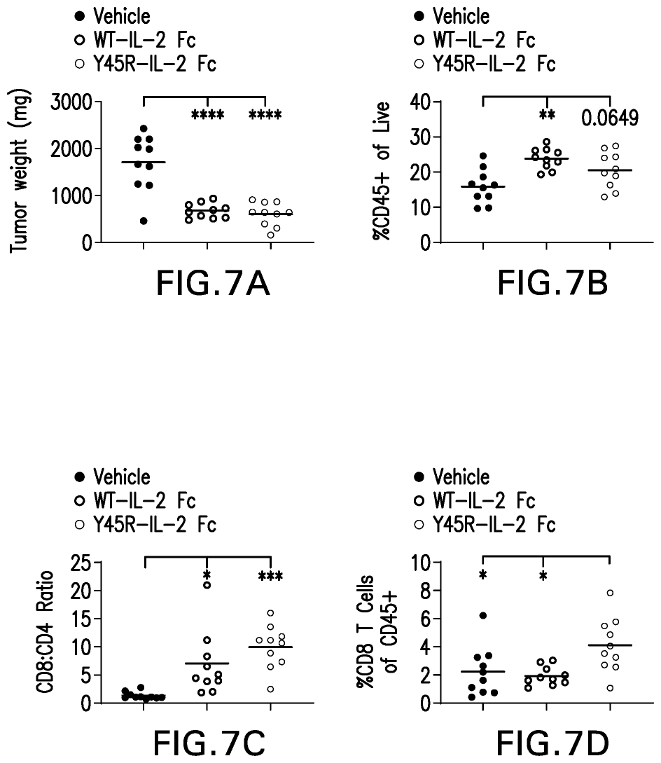

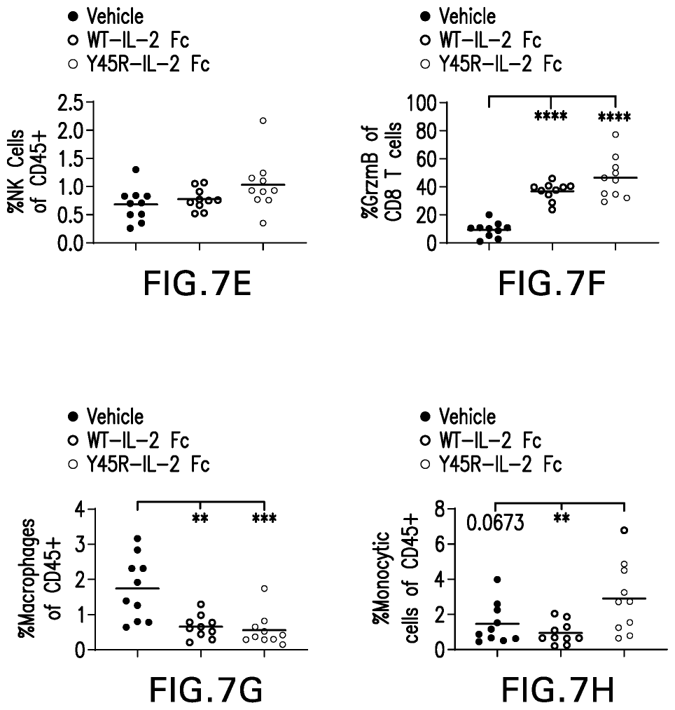

The pharmacodymanic effects of Y45R-IL-2 Fc on immune cell subtypes were assessed in B16F10 tumor-bearing mice in both the tumor microenvironment (TME) and periphery. Mice bearing B16F10 tumors (˜100 mm³) were administered a single doses of 0.23 mg/kg IL-2 equivalents of WT-IL-2 Fc or Y45R-IL-2 Fc. Flow cytometry analysis was performed on tumors, blood and spleen on day 7 after treatment initiation. On day 7, tumors from mice treated with WT-IL-2 Fc and Y45R-IL-2 Fc were smaller by weight than vehicle treated controls (FIG. 7A) but had an increased percentage of CD45⁺ immune cell infiltration (FIG. 7B). Of immune cells, the percentage of T cells was increased in the TME of treated mice, and this was more prominent in the CD8⁺ T cell subset over the CD4⁺ T cells (FIG. 7C). Specifically, the percentage of CD8⁺ T cells was increased in tumors of mice treated with Y45R-IL-2 Fc over those of vehicle or WT-IL-2 Fc treated mice (FIG. 7D). While there was no difference in the percentage of NK cell infiltration in treated tumors (FIG. 7E), there was an increased percentage of Granzyme B positive CD8⁺ T cells and NK cells in the tumors of WT-IL-2 Fc and Y45R-IL-2 Fc treated mice suggesting cytolytic function (FIG. 7F). In addition to the lymphocytic cell subsets, the myeloid populations were examined and there was a decrease in the percentage of macrophage infiltration, both MHCII⁺ and MHCII-low expressing populations (FIG. 7G) as well as on increase in MHCII⁺ monocytic cells in mice treated with Y45R-IL-2 Fc and dendritic cells in mice treated with WT-IL-2 Fc (FIG. 7H).

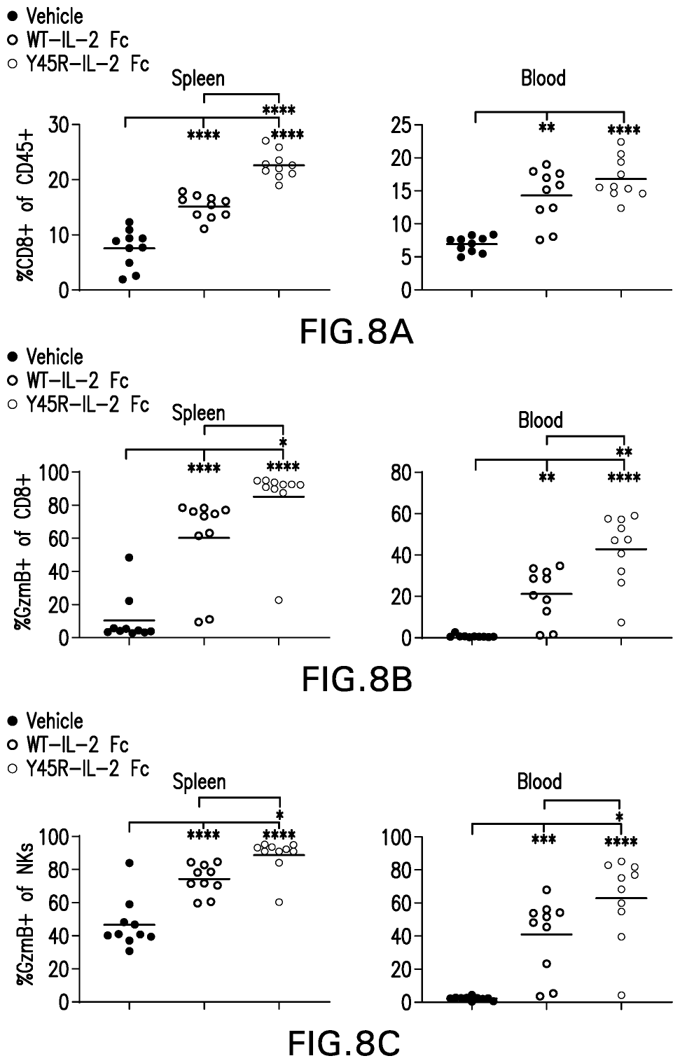

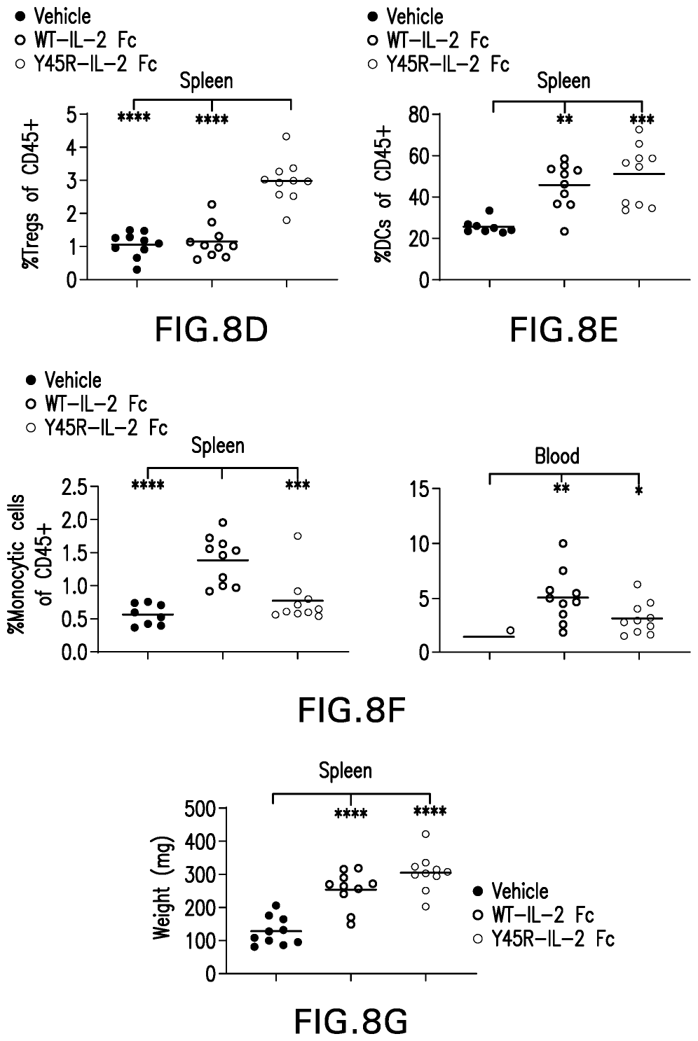

Given the systemic administration of these compounds, peripheral immune cell changes were examined in the blood and spleen of treated mice. As observed in the TME, there was an increased percentage of CD8⁺ T cells in the spleen and blood of mice treated with both WT-IL-2 Fc and Y45R-IL-2 Fc, and this increase was higher in mice treated with Y45R-IL-2 Fc (FIG. 8A). There was also an increased percentage of granzyme B⁺CD8⁺ T cells (FIG. 8B) and NK cells (FIG. 8C) in the spleen and the blood of mice treated with WT-IL-2 Fc, and this increase was more striking in mice treated with Y45R-IL-2 Fc. There was also an increased percentage of Tregs in the spleen of mice treated with Y45R-IL-2 Fc (FIG. 8D). As observed in the tumor microenvironment, there were also changes in the myeloid cells in the periphery. There was an increase percentage of dendritic cells found in the spleens of mice treated with both WT-IL-2 Fc and Y45R-IL-2 Fc (FIG. 8E) as well as increase in MHCII⁺ monocytic cells in the spleen (WT-IL-2 Fc) and blood (WT-IL-2 Fc and Y45R-IL-2 Fc) (FIG. 8F). Due to the number of immune cell subsets expanded in the spleen, mice treated with both WT-IL-2 Fc and Y45R-IL-2 Fc displayed splenomegaly (FIG. 8G).

### EXAMPLE 9. CHARACTERIZATION OF Y45R-IL-2 FC IN CT26, B16F10 AND EMT6 MOUSE SYNGENEIC TUMOR MODELS

The efficacy of Y45R-IL-2 Fc was evaluated in multiple syngeneic mouse tumor models including EMT6, B16F10 and CT26. Mice bearing established tumors (˜100 mm³) were treated with 1 mg/kg Y45R-IL-2 Fc or aldesleukin at the dosing schedule indicated in Table 6. Doses used in the efficacy studies were selected based on previously established maximum tolerated dose (MTD) levels (data not shown).

TABLE 6
Study design in EMT6, B16F10 and CT26 syngeneic mouse tumor models

| Treatment | Dose [mg/kg] (ROA) | Dosing schedule |
|---|---|---|
| Vehicle pH5.5 + | NA (IP) | Q5D×4 |
| 9% Sucrose | | |
| Y45R-IL-2 Fc | 1 (IP) | Q5D×4 |
| Aldesleukin | 1 (IP) | BID 5D, 2D off ×2 |

Summary of treatment, dose level, route of administration [ROA]: Intraperitoneal [IP], Intravenous [IV] and dosing schedule of individual compounds. Q5D×4 [Once daily on day 0, 5, 10, and 15]; Q7D×3 [Once daily on day 0, 7, and 14]; BID 5D, 2D off ×2 [Twice daily on day 0, 1, 2, 3, 4, two days off, and twice daily on day 7, 8, 9, 10 and 11].

Treatment with Y45R-IL-2 Fc at 1 mg/kg dose resulted in various efficacy levels (˜69%, 80% or 47% tumor growth inhibition [TGI]) compared to vehicle control in EMT6, B16F10 and CT26, respectively (Table 7). Treatment with aldesleukin at 1 mg/kg dose resulted in significant efficacy of 75% TGI compared to vehicle control in CT26 (Table 7). All treatments were well tolerated with little or no body weight loss (data not shown). Summary of complete responses [CR] assessed at the study completion and tumor growth inhibition [TGI] calculated on Day 17 (EMT6), Day 10 (B16F10) or Day 14 (CT26).

TABLE 7
Results in EMT6, B16F10 and CT26 syngeneic mouse tumor models

| | EMT6 | B16F10 | CT26 |
|---|---|---|---|
| | Tumor Growth | Tumor Growth | Tumor Growth |
| | Inhibition [TGI] | Inhibition [TGI] | Inhibition [TGI] |
| | Complete | Complete | Complete |
| | Responses | Responses | Responses |
| Treatment | [CR] | [CR] | [CR] |
| Y45R-IL-2 Fc | 69% TGI | 80%/1 CR | 47% TGI |
| Aldesleukin | — | — | 75% TGI |

### EXAMPLE 10. COMBO STUDIES WITH ANTI-PD-1 ANTIBODY DX400 IN THE CT26 SYNGENEIC TUMOR MODEL

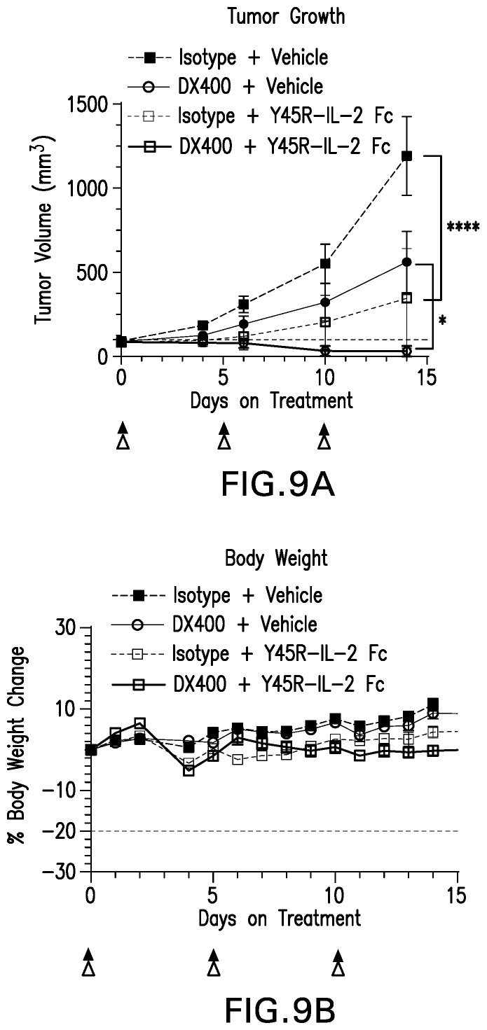

The efficacy of Y45R-IL-2 Fc as a single agent and in combination with anti-PD-1 antibody, DX400, was evaluated in the CT26 mouse syngeneic tumor model. Mice bearing established CT26 tumors (˜100 mm³) were treated with 1 mg/kg Y45R-IL-2 Fc and 10 mg/kg DX400 at the dosing schedule indicated in Table 8. Treatment with Y45R-IL-2 Fc resulted in significant efficacy (˜76% TGI, p<0.0001) compared to vehicle control (FIG. 9A). Although not statistically significant, the dose of 10 mg/kg DX400 in combination with Y45R-IL-2 Fc demonstrated improved efficacy compared to Y45R-IL-2 Fc dosed as a single agent (105% TGI vs 76% respectively) (FIG. 9A). Both treatments were well tolerated with little or no body weight loss (FIG. 9B).

Aldesleukin was evaluated in parallel. Treatment with aldesleukin at 1 mg/kg dose at dosing schedule indicated below resulted in efficacy (˜54% TGI) compared to vehicle control (Table 8). Efficacy of aldesleukin was enhanced by DX400. The dose of 10 mg/kg DX400 in combination with aldesleukin demonstrated improved efficacy compared to aldesleukin dosed as a single agent (95% TGI vs 54% respectively).

TABLE 8
Y45R-IL-2 Fc and aldesleukin as single agents or in combination
with DX400 in the CT26 mouse syngeneic tumor model

| | | | Tumor | |
|---|---|---|---|---|
| | | | Growth | Complete |
| | Dose | | Inhibition | Responses |
| | [mg/kg] | Dosing | [TGI] on | [CR] on |
| Treatment | (ROA) | schedule | Day 14 | Day 21 |
| Isotype + | NA (IP) | Q5D×4 + Q5D×4 | NA | 0 CR |
| Vehicle | | | | |
| D×400 + | 10 (IP) + NA | Q5D×4 + Q5D×4 | 57% TGI | 3 CR |
| Vehicle | | | | |
| Isotype + | NA (IP) + 1 (IP) | Q5D×4 + Q5D×4 | 76% TGI | 1 CR |
| Y45R-IL-2 | | | | |
| Fc | | | | |
| D×400 + | 10 (IP) + 1 (IP) | Q5D×4 + Q5D×4 | 105% TGI | 5 CR |
| Y45R-IL-2 | | | | |
| Fc | | | | |
| Isotype + | NA (IP) + 1 (IP) | Q5D×4 + BID | 54% TGI | 1 CR |
| Aldesleukin | | 5D, 2D off ×2 | | |
| D×400 + | 10 (IP) + 1 (IP) | Q5D×4 + BID | 95% TGI | 3 CR |
| Aldesleukin | | 5D, 2D off ×2 | | |

Tumor growth inhibition [TGI] calculated on Day 14 in response to indicated doses of Y45R-IL-2 Fc and Aldesleukin in combination with DX400 administered intraperitoneally q5d—3. Complete responses [CR] assessed on Day 21. Route of administration [ROA]: Intraperitoneal [IP], Intravenous [IV]. Dosing schedule Q5D—4 [Once daily on day 0, 5, 10, and 15]; Q7D—3 [Once daily on day 0, 7, and 14]; BID 5D, 2D off —2 [Twice daily on day 0, 1, 2, 3, 4, two days off, and twice daily on day 7, 8, 9, 10 and 11].

All references cited herein are incorporated by reference to the same extent as if each individual publication, database entry (e.g. GenBank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. GenBank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. An IL-2 mutein comprising a first polypeptide comprising an amino acid sequence as set forth in SEQ ID NO: 3, 4, 18, or 19.

2. The IL-2 mutein of claim 1, wherein the first polypeptide further comprises an amino acid sequence as set forth in SEQ ID NO: 5, 6, or 7.

3. The IL-2 mutein of claim 2, wherein the first polypeptide further comprises a linker as set forth in SEQ ID NO: 8, 15, or 16.

4. The IL-2 mutein of claim 2, further comprising a second polypeptide comprising an amino acid sequence as set forth in SEQ ID NO:9 or SEQ ID NO:10.

5. The IL-2 mutein of claim 2, further comprising a second polypeptide, wherein
(1) the first polypeptide comprises an amino acid sequence as set forth in SEQ ID NO:11 and the second polypeptide comprises an amino acid sequence as set forth in SEQ ID NO:10;
(2) the first polypeptide comprises an amino acid sequence as set forth in SEQ ID NO:12 and the second polypeptide comprises an amino acid sequence as set forth in SEQ ID NO:9;
(3) the first polypeptide and the second polypeptide both comprise an amino acid sequence as set forth in SEQ ID NO:17;
(4) the first polypeptide comprises an amino acid sequence as set forth in SEQ ID NO:20 and the second polypeptide comprises an amino acid sequence as set forth in SEQ ID NO:10;
(5) the first polypeptide comprises an amino acid sequence as set forth in SEQ ID NO:21 and the second polypeptide comprises an amino acid sequence as set forth in SEQ ID NO:9; or
(6) the first polypeptide and the second polypeptide both comprise an amino acid sequence as set forth in SEQ ID NO:22.

6. An IL-2 mutein comprising a first polypeptide and a second polypeptide, wherein the first polypeptide comprises an amino acid sequence as set forth in SEQ ID NO:11; and the second polypeptide comprises an amino acid sequence as set forth in SEQ ID NO:10.

7. An IL-2 mutein consisting of a first polypeptide and a second polypeptide, wherein the first polypeptide consists of an amino acid sequence as set forth in SEQ ID NO:11; and the second polypeptide consists of an amino acid sequence as set forth in SEQ ID NO:10.

8. A pharmaceutical composition comprising the IL-2 mutein of claim 6, and a pharmaceutically acceptable carrier.

9. A method of treating an IL-2-mediated disease in a subject, comprising administering to the subject a therapeutically effective amount of the IL-2 mutein of claim 6.

10. The method of claim 9, wherein the IL-2-mediated disease is cancer.

11. The method of claim 9, wherein the IL-2-mediated disease is infection.

12. The method of claim 10, wherein the cancer is osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer (NSCLC), gastric cancer, colorectal cancer, cervical cancer, synovial sarcoma, head and neck cancer, squamous cell carcinoma, lymphoma, diffuse large B-cell lymphoma (DLBCL), non-Hodgkin lymphoma (NHL), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, or carcinoid cancer.

13. The method of claim 10, wherein the cancer is advanced renal cell carcinoma or metastatic melanoma.

14. A method of selectively activating CD8+ T cells or NK cells without significantly activating T regulatory cells in a subject, comprising administering to a subject in need thereof a therapeutically effective amount of the IL-2 mutein of claim 6.

15. A method of selectively activating cells that express IL-2 receptor βγ subunit but without significantly activating cells that express IL-2 receptor αβγ subunit compared to wild type IL-2 in a subject, comprising administering to a subject in need thereof a therapeutically effective amount of the IL-2 mutein of claim 6.

16. An isolated nucleic acid, encoding:
(a) a first polypeptide comprising the IL-2 mutein of SEQ ID NO: 11;
(b) a second polypeptide comprising the IL-2 mutein of SEQ ID NO: 10; or
(c) the first and the second polypeptides comprising the IL-2 muteins of SEQ ID NO:11 and SEQ ID NO: 105.

17. An expression vector comprising the isolated nucleic acid of claim 16.

18. A host cell comprising the expression vector of claim 17.

19. A method of producing an IL-2 mutein, comprising:
culturing the host cell of claim 18
under conditions wherein the IL-2 mutein is expressed.

20-24. (canceled)

25. The method of claim 9, further comprising administering to the subject a second agent.

26. The method of claim 25, wherein the second agent is a PD-1 antagonist, a PD-L1 antagonist, a CTLA4 antagonist, a LAG3 antagonist, a TIGIT antagonist, an ILT3 antagonist, an ILT4 antagonist, a PARP antagonist, a VEGF receptor antagonist, a FGF receptor antagonist, a HIF-2α antagonist, a BTK antagonist, an AKT antagonist, an ERK antagonist, a MEK antagonist, a CD27 agonist, a STING agonist, a chemotherapeutic agent, or an antibody-drug conjugate.

27. The method of claim 26, wherein the second agent is a PD-1 antagonist.

28. The method of claim 27, wherein the PD-1 antagonist is pembrolizumab, nivolumab, cemiplimab, or pidilizumab.

29. The method of claim 28, wherein the PD-1 antagonist is pembrolizumab.

30. The method of claim 26, wherein the second agent is a PD-L1 antagonist.

31. The method of claim 30, wherein the PD-L1 antagonist is atezolizumab, durvalumab, or avelumab.

32-42. (canceled)

43. The method of claim 25, wherein the IL-2 mutein and the second agent are administered simultaneously.

44. The method of claim 25, wherein the IL-2 mutein and the second agent are administered sequentially.

45. The method of claim 25, wherein the IL-2 mutein and the second agent are administered through the same routes.

46. The method of claim 25, wherein the IL-2 mutein and the second agent are administered through different routes.
